(19) United States
(12) Patent Application Publication
(10) Pub. No.: US 2024/0026359 A1
(43) Pub. Date: Jan. 25, 2024

(54) Methods of Treating of Spinal Stenosis and Ligamentum Flavum Hypertrophy

(71) Applicants: University of Pittsburgh-Of the Commonwealth System of Higher Education, Pittsburgh, PA (US); UPMC, Pittsburgh, PA (US)

(72) Inventors: Joon Lee, Venetia, PA (US); Gwendolyn A. Sowa, Pittsburgh, PA (US); Nam V. Vo, Pittsburgh, PA (US); Richard Wawrose, Pittsburgh, PA (US)

(21) Appl. No.: 18/354,358

(22) Filed: Jul. 18, 2023

Related U.S. Application Data
(60) Provisional application No. 63/390,494, filed on Jul. 19, 2022.

(51) Int. Cl. C12N 15/113

(52) U.S. Cl. CPC C12N 15/113; C12N 2310/141

(57) ABSTRACT

Provided herein are methods of treating ligament flavum hypertrophy and spinal stenosis including ligament flavum hypertrophy in a patient. The method comprises delivering to cells of hypertrophic ligament flavum in a patient a pharmaceutical composition comprising micro-RNA 29a or a precursor thereof, thereby decreasing type I and/or III collagen production by the cells. Alternatively, an antisense or RNAi reagent for knocking down type I collagen and/or type III collagen expression is delivered to cells of hypertrophic ligament flavum in a patient.

### CROSS REFERENCE TO RELATED APPLICATIONS

This application claims priority to U.S. Provisional Patent Application No. 63/390,494 filed Jul. 19, 2022, the disclosure of which is incorporated herein by reference in its entirety.

The Sequence Listing associated with this application is filed in electronic format via Patent Center and is hereby incorporated by reference into the specification in its entirety. The name of the XML file containing the Sequence Listing is 2302963.xml. The size of the XML file is 18,485 bytes and the XML file was created on Jul. 11, 2023.

Spinal stenosis of the lumbar spine has numerous etiologies, but a significant contributor is ligamentum flavum hypertrophy. Spinal stenosis may have been described in the academic literature as early as 1900. Despite noticeable advancements in treatments since that era, there is still considerable room for improvement. As recently as 2007, laminectomy as an operative intervention was attributable to $1.65 billion in Medicare costs. It is the most common reason for spinal surgery in patients over the age of 65. Current evidence suggests non-operative measures have mixed efficacies. Therefore, the development of novel treatment or preventative modalities to decrease overall cost and morbidity associated with spinal stenosis is of great importance and interest.

### SUMMARY

A method of treating spinal stenosis in a patient is provided. The method comprises delivering to cells of a patient's ligamentum flavum (LF), e.g., transfecting cells of the patient's LF with, an amount of a MIR29A reagent effective to treat spinal stenosis in the patient.

In a further aspect or embodiment, a method of reducing expression of type I and/or III collagen, and/or treating fibrosis in LF of a patient also is provided. The method comprises delivering to cells of a patient's ligamentum flavum (LF), e.g., transfecting cells of the patient's LF with, an amount of a MIR29A reagent effective to reduce expression of type I and/or III collagen, and/or treat fibrosis in LF in the patient.

In another aspect or embodiment, a method of treating hypertrophic LF in a patient is provided. The method comprises delivering to cells of a patient's ligamentum flavum (LF), e.g., transfecting cells of the patient's LF with, an amount of a MIR29A reagent effective to treat hypertrophic LF in the patient.

In yet another aspect or embodiment, a method of treating spinal stenosis in a patient is provided. The method comprises delivering to cells of a patient's ligamentum flavum (LF), e.g., transfecting cells of the patient's LF with, an amount of a reagent for knocking down type I collagen expression in the cells effective to treat spinal stenosis in the patient and/or an amount of a reagent for knocking down type III collagen expression in the cells effective to treat spinal stenosis in the patient.

In a further aspect or embodiment, a method of reducing expression of type I and/or treating fibrosis in LF of a patient is provided. The method comprises delivering to cells of a patient's ligamentum flavum (LF), e.g., transfecting cells of the patient's LF with, an amount of a reagent for knocking down type I collagen expression in the cells effective to reduce expression of type I collagen in the cells, and/or treat fibrosis in LF in the patient and/or an amount of a reagent for knocking down type III collagen expression in the cells effective to reduce expression of type III collagen in the cells, and/or treat fibrosis in LF in the patient.

In another aspect or embodiment, a method of treating hypertrophic LF in a patient is provided. The method comprises delivering to cells of a patient's ligamentum flavum (LF), e.g., transfecting cells of the patient's LF with, an amount of a reagent for knocking down type I collagen expression in the cells effective to treat hypertrophic LF in the patient and/or an amount of a reagent for knocking down type III collagen expression in the cells effective to treat hypertrophic LF in the patient.

The following numbered clauses describe certain exemplary aspects or embodiments of the present invention:

Clause 1. A method of treating spinal stenosis in a patient, comprising delivering to cells of a patient's ligamentum flavum (LF) an amount of a MIR29A reagent effective to treat spinal stenosis in the patient.

Clause 2. A method of reducing expression of type I and/or III collagen, and/or treating fibrosis in LF of a patient, comprising delivering to cells of a patient's ligamentum flavum (LF) an amount of a MIR29A reagent effective to reduce expression of type I and/or III collagen, and/or treat fibrosis in LF in the patient.

Clause 3. The method of clause 1 or 2, wherein the patient has hypertrophic LF, and the MIR29A reagent is delivered to cells of the hypertrophic LF.

Clause 4. A method of treating hypertrophic LF in a patient, comprising delivering to cells of a patient's ligamentum flavum (LF) an amount of a MIR29A reagent effective to treat hypertrophic LF in the patient.

Clause 5. The method of clause 4, wherein the MIR29A reagent is delivered to cells of the hypertrophic LF

Clause 6. The method of any one of clauses 1-5, wherein the MIR29A reagent is delivered by transforming a cell of the LF of the patient with a nucleic acid comprising a gene for expressing the MIR29A reagent.

Clause 7. The method of clause 6, wherein the nucleic acid is a recombinant viral genome, optionally delivered to the cell in a viral particle.

Clause 8. The method of clause 7, wherein the recombinant viral genome is an Adeno-associated Virus (AAV) genome, optionally packaged in a viral transducing unit.

Clause 9. The method of clause 6, wherein the nucleic acid is delivered to a cell of the LF of the patient in a liposome particle.

Clause 10. The method of any one of clauses 1-9, wherein the MIR29A reagent is conspecific to the patient.

Clause 11. The method of any one of clauses 1-10, wherein the MIR29A reagent is delivered in a pharmaceutical composition by a nanocarrier.

Clause 12. The method of clause 11, wherein the nanocarrier is a liposome, a lipid nanoparticle, an exosome, a dendrimer, or a polymer particle.

Clause 13. The method of clause 11, wherein the nanocarrier is a lipid nanoparticle.

Clause 14. The method of clause 13, wherein the lipid nanoparticle comprises comprising the MIR29A reagent, an ionizable lipid, a helper lipid, a PEGylated lipid, and a cholesterol.

Clause 15. The method of any one of clauses 1-14, wherein the LF is injected directly with the MIR29A reagent by epidural delivery.

Clause 16. The method of clause 15, wherein the epidural delivery is performed with image guidance, for example by X-ray or computer tomography-assisted (CT-assisted) delivery.

Clause 17. The method of clause 15, wherein the epidural delivery is via one or more injections.

Clause 18. The method of clause 15, wherein the epidural delivery is via an implanted catheter, for example by multiple or continuous infusion.

Clause 19. The method of any one of clauses 1-18, wherein the MIR29A reagent is a microRNA-29a precursor RNA.

Clause 20. The method of clause 19, wherein the microRNA-29a precursor has the sequence: augacugauuucuuuugguguucagagucaauauaauuuucuagcaccaucugaaaucgguuau (SEQ ID NO: 21).

Clause 21. The method of clause 19, wherein the microRNA-29a precursor is an allele, variant, or homolog of human microRNA-29a effective to reduce type I or III collagen expression in an LF cell of the patient.

Clause 22. The method of any one of clauses 1-18, wherein the MIR29A reagent is a mature microRNA-29a RNA.

Clause 23. The method of clause 22, wherein the MIR29A reagent comprises either one or both of:
hsa-miR-29a-5p MIMAT0004503: 5′-acugauuucuuuugguguucag-3′ (SEQ ID NO: 3); and
hsa-miR-29a-3p MIMAT0000086: 5′-uagcaccaucugaaaucgguua-3′ (SEQ ID NO: 4).

Clause 24. A method of treating spinal stenosis in a patient, comprising delivering to cells of a patient's ligamentum flavum (LF), e.g., transfecting cells of the patient's LF with, an amount of a reagent for knocking down type I collagen expression in the cells effective to treat spinal stenosis in the patient and/or an amount of a reagent for knocking down type III collagen expression in the cells effective to treat spinal stenosis in the patient.

Clause 25. A method of reducing expression of type I collagen, expression or type III collagen, and/or treating fibrosis in LF of a patient, comprising delivering to cells of a patient's ligamentum flavum (LF), e.g., transfecting cells of the patient's LF with, an amount of a reagent for knocking down type I collagen expression in the cells effective to reduce expression of type I collagen in the cells, and/or treat fibrosis in LF in the patient and/or an amount of a reagent for knocking down type III collagen expression in the cells effective to reduce expression of type III collagen in the cells, and/or treat fibrosis in LF in the patient.

Clause 26. The method of clause 24 or 25, wherein the patient has hypertrophic LF, and the reagent is delivered to cells of the hypertrophic LF.

Clause 27. A method of treating hypertrophic LF in a patient, comprising delivering to cells of a patient's ligamentum flavum (LF), e.g., transfecting cells of the patient's LF with, an amount of a reagent for knocking down type I collagen expression in the cells effective to treat hypertrophic LF in the patient and/or an amount of a reagent for knocking down type III collagen expression in the cells effective to treat hypertrophic LF in the patient.

Clause 28. The method of clause 27, wherein the reagent is delivered to cells of the hypertrophic LF.

Clause 29. The method of any one of clauses 24-28, wherein the reagent is conspecific to the patient.

Clause 30. The method of any one of clauses 24-29, wherein the reagent is delivered in a pharmaceutical composition by a nanocarrier.

Clause 31. The method of clause 30, wherein the nanocarrier is a liposome, a lipid nanoparticle, an exosome, a dendrimer, or a polymer particle.

Clause 32. The method of clause 30, wherein the nanocarrier is a lipid nanoparticle.

Clause 33. The method of clause 32, wherein the lipid nanoparticle comprises comprising the reagent, an ionizable lipid, a helper lipid, a PEGylated lipid, and a cholesterol.

Clause 34. The method of any one of clauses 24-33, wherein the LF is injected directly with the reagent by epidural delivery.

Clause 35. The method of clause 34, wherein the epidural delivery is performed with image guidance, for example by X-ray or computer tomography-assisted (CT-assisted) delivery.

Clause 36. The method of clause 34, wherein the epidural delivery is via one or more injections.

Clause 37. The method of clause 34, wherein the epidural delivery is via an implanted catheter, for example by multiple or continuous infusion.

Clause 38. The method of any one of clauses 24-34, wherein the reagent is an antisense reagent.

Clause 39. The method of any one of clauses 24-34, wherein the reagent is an RNAi reagent.

Clause 40. The method of clause 39, wherein the reagent is an siRNA.

Clause 41. The method of any one of clauses 1-40, wherein the patient is a human patient.

### BRIEF DESCRIPTION OF THE DRAWINGS

FIG. 1 depicts an exemplary stem-loop structure for a MIR29A reagent, with mature sequences highlighted in gray (see below, SEQ ID NO: 2).

FIG. 2 is a graph showing microRNA-29a and collagen I relative gene expression profiles in patients with hypertrophic ligamentum flavum as compared to control.

FIG. 3 is a microRNA-29a Pearson correlation plot depicting relationship with Collagen I mRNA gene expression profile in cells of hypertrophic ligamentum flavum tissue.

FIG. 4 is a photomicrograph of an exemplary Ligamentum Flavum Cell Culture as visualized under brightfield light microscopy (bar is 50 μm).

FIG. 5: MicroRNA-29a lentiviral plasmid precursor restriction enzyme map created using SnapGene® molecular cloning tool. MicroRNA-29a gene is depicted in the bottom right of the image.

FIG. 6: Successful Transfection of Ligamentum Flavum Cells in Culture with a Plasmid Containing the Green fluorescent protein (GFP) Reporter. GFP-illuminated cells indicate successful transfected cells. Ligamentum flavum cells will be transfected with plasmids containing sequences for upregulating or downregulating microRNA-29a gene expression.

FIGS. 7A-7D are graphs showing Collagen I expression (FIGS. 7A and 7B) expression or collagen III expression (FIGS. 7C and 7D) in LF cells transduced with a microRNA-29a overexpressor (FIGS. 8A and 8C) microRNA-29a inhibitor (FIGS. 8B and 8D).

FIGS. 8A and 8B together, contiguously, provide an exemplary human type I collagen cDNA sequence (SEQ ID NO: 6). FIGS. 8C and 8D together, contiguously, provide an exemplary human type III collagen cDNA sequence (SEQ ID NO: 7).

FIG. 9. 3D LF Model macroscopic appearance. LF model cultured in Tissue-Train™ plates with (A-C) TGFb or (D-F) IL1b for (A&D) 3 days or (B&C, D&E) 8 days that includes exposure to (B&C) TGFb or (E&F) IL1b treatment for the final 24 hours. At day 8, most samples treated with were still contiguous (B&E). After 24 hours of TGFb treatment, 2/12 samples had ruptured (C) as compared to 6/12 rupturing with 24 hours of IL1b treatment.

FIGS. 10A-10C: Gene Expression Analysis of Monolayer (2D) Cultures. *p<0.05.

FIGS. 11A-11C. Gene Expression Analysis of Hydrogel (3D) Models. *p<0.05.

FIG. 12: ECM Elastin production in healthy and hypertrophic LF tissue. Histological staining of Elastin by Verhoeff s elastic stain in (A) normal and (B) hypertrophic human LF tissue changed in bipedal standing mouse and human LF. The elastic fibers stain purple-black (in original) and the collagen fibers stain red (in original). (C) Quantitative analyses of the ratio of elastic fibers to collagen fibers in human LF (n=8). Adapted from: Sun C, et al. Ligamentum flavum fibrosis and hypertrophy: Molecular pathways, cellular mechanisms, and future directions. FASEB J. 2020 August; 34(8):9854-9868.

FIG. 13. Schematic representation of rat spine instability model. (A) Rat spine from L2-L4; (B) Destabilized rat spine from L2-L4. Labels: SAP, superior articular process; TP, Transverse process; VB, vertebral body; SP, spinous process; PI, pars interarticularis; IAP, inferior particular process; SAP, superior articular process; LF, ligamentum flavum; ISL, interspinous ligament; ISP L3-R, inferior spinous process of L3 resected; SSP L4-R, superior spinous process of L4 resected; ISL-R, Interspinous ligament resected; PI/IAP L3-R Pars interarticularis/Inferior articular process of L3 resected; SAP L4-R, superior articular process of L4 resected.

### DETAILED DESCRIPTION

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of these ranges is intended as a continuous range including every value between the minimum and maximum values. For definitions provided herein, those definitions also refer to word forms, cognates and grammatical variants of those words or phrases.

As used herein, the terms “comprising,” “comprise”, or “comprised,” and variations thereof, in reference to elements of an item, composition, apparatus, method, process, system, claim etc. are intended to be open-ended, meaning that the item, composition, apparatus, method, process, system, claim etc. includes those elements and other elements can be included and still fall within the scope/definition of the described item, composition, apparatus, method, process, system, claim etc. As used herein, “a” or “an” means one or more. As used herein “another” may mean at least a second or more.

As used herein, the terms “patient” or “subject” refer to members of the animal kingdom, including, but not limited to human beings.

As used herein, a “pharmaceutically acceptable excipient”, “aqueous carrier” or “pharmaceutically acceptable aqueous carrier” refer to solvents or dispersion media, and the like, that are physiologically compatible. Examples of pharmaceutically acceptable carriers include one or more of water, saline, phosphate buffered saline, dextrose, glycerol, ethanol, and the like, as well as combinations thereof. Pharmaceutically acceptable carriers may further comprise minor amounts of auxiliary substances such as wetting or emulsifying agents, preservatives or buffers, which enhance the shelf life or effectiveness of the active agent. Such excipients or carriers, include vehicles for delivering RNA as described herein, such as, for example and without limitation, LNPs, liposomes, or other nanoparticle.

An “effective amount” or “amount effective” to achieve a desirable therapeutic, pharmacological, medicinal, or physiological effect is any amount that achieves the stated purpose. For example, an amount of the MIR29A reagent effective to reduce collagen I or III expression in LF of a patient, and/or to treat LF hypertrophy or spinal stenosis in a patient. Based on the teachings provided herein, one of ordinary skill can readily ascertain effective amounts of the elements of the described dosage form and produce a safe and effective dosage form and drug product. Examples of an effective amount of the MIR29A reagent compounded in a delivery vehicle-containing composition includes from 100 μg per ml (picograms per milliliter) to 1 mg/ml (milligrams per milliliter) of solution, including any increment therebetween, such as from 1 ng/ml (nanogram/milliliter) to 1 mg/ml or from 1 ng/ml to 1 μg/ml (microgram/milliliter). Equivalent amounts, including molar or w/v (weight/volume) equivalents, of other MIR29A reagents may be utilized in the methods described herein.

The term “homology” can refer to a percent (%) identity of an RNA to a reference RNA. As a practical matter, whether any particular RNA can be at least 50%, 60%, 70%, 80%, 85%, 90%, 92%, 95%, 96%, 97%, 98%, or 99% identical to any reference RNA sequence of any RNA described herein (which may correspond with a particular nucleic acid sequence described herein), such particular RNA sequence can be determined conventionally using known computer programs such the Bestfit program (Wisconsin Sequence Analysis Package). When using Bestfit or any other sequence alignment program to determine whether a particular sequence is, for instance, 95% identical to a reference sequence according to the present invention, the parameters can be set such that the percentage of identity is calculated over the full length of the reference RNA sequence and that gaps in homology of up to 5% of the total number of RNA bases in the reference sequence are allowed.

MicroRNAs (miRNAs) are short (20-24 nt) non-coding RNAs that are involved in post-transcriptional regulation of gene expression in multicellular organisms by affecting both the stability and translation of mRNAs. miRNAs are transcribed by RNA polymerase II as part of capped and polyadenylated primary transcripts (pri-miRNAs) that can be either protein-coding or non-coding. The primary transcript is cleaved by the Drosha ribonuclease III enzyme to produce an approximately 70-nt stem-loop precursor miRNA (pre-miRNA), which is further cleaved by the cytoplasmic Dicer ribonuclease to generate the mature miRNA and antisense miRNA star (miRNA*) products. The mature miRNA is incorporated into a RNA-induced silencing complex (RISC), which recognizes target mRNAs through imperfect base pairing with the miRNA and most commonly results in translational inhibition or destabilization of the target mRNA.

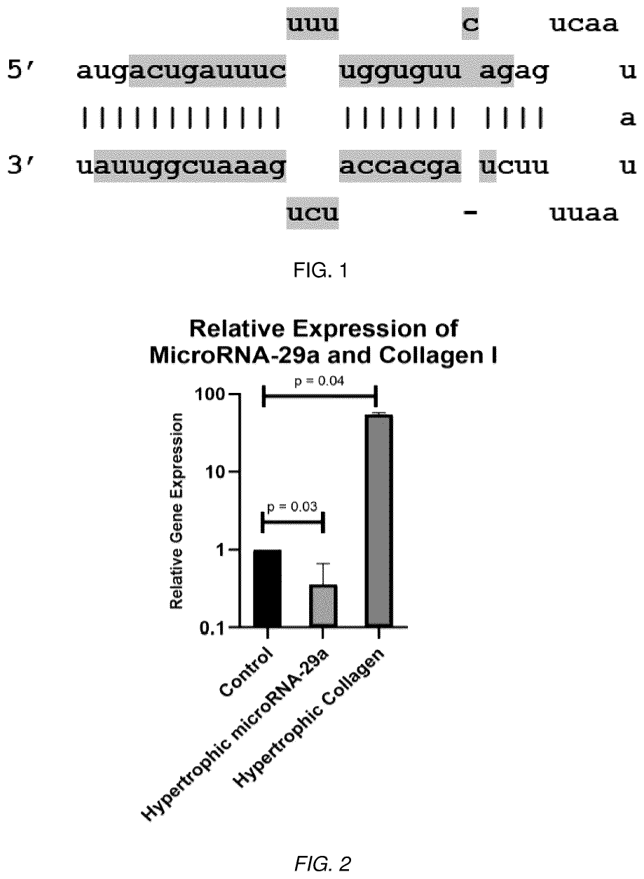

microRNA 29a (MIR29A) is abundantly-described in the literature. Exemplary references include: NCBI Reference Sequence: NR 029503.1, Gene ID: 407021, miRBase accession No. MI0000087 (mirbase.org, See, e.g., FIG. 1), and OMIM Ref. No. 610782. The following is an exemplary (DNA) sequence of Homo sapiens MIR29A Pre-miRNA. Bases 4-25, and 42-63 are ncRNAs (non-coding RNAs):

>NR_029503.1 Homo sapiens microRNA 29a (MIR29A), microRNA
(SEQ ID NO: 1, cDNA)
5′-atgactgatttcttttggtgttcagagtcaatataattttctagcaccatctgaaatcggttat-3′,
with the corresponding RNA having the sequence:
(SEQ ID NO: 2)
5′-augacugauuucuuuugguguucagagucaauauaauuuucuagcaccaucugaaaucgguuau-3′.

Mature RNA sequences include, for example:

hsa-miR-29a-5p MIMAT0004503:
(SEQ ID NO: 3)
5′-acugauuucuuuugguguucag-3′;
and
hsa-miR-29a-3p MIMAT0000086:
(SEQ ID NO: 4)
5′-uagcaccaucugaaaucgguua-3′.

Numerous human sequence variants are known, e.g., based on hsa-miR-29a-5p MINIAT0004503 (SEQ ID NO: 3), variants include, without limitation: rs1380872787 (A/G), rs1419063786 (A/G), and rs782312117 (A/G), and based on hsa-miR-29a-3p MIMAT0000086 (SEQ ID NO: 4), variants include, without limitation: rs1554489270 (G/A) and rs1799635468 (C/T), as shown in FIG. 2 of priority U.S. provisional patent application No. 63/390,494 filed Jul. 19, 2023, which is hereby incorporated herein by reference in its entirety. Referring to FIG. 1, it is noted that the sequence of the a MIR29A nucleic acid reagent may include natural variants, so long as appropriate sequences are present to form any necessary intermediate structures or final active reagents (e.g., pri-miRNA, pre-miRNA, or mature miRNA) by cellular processing to effectively knock down expression of Col I and/or Col III mRNA. It may be helpful to sequence a patient's M1R29, COLI, and/or COLIIIA genes to determine an optimal MIR29A sequence to match the specific patient's sequences. Any changes to the sequence of one strand of the RNA that forms the hairpin of pri-miRNA or pre-miRNA may be represented in the other strand of the hairpin to ensure proper hairpin formation.

By “a microRNA 29a RNA”, “a MIR29A RNA”, “a microRNA 29a RNA reagent”, “a MIR29A RNA”, “a MIR29A reagent”, or the like, it is meant an RNA or analog thereof having a nucleic acid sequence of MIR29A, or an analog thereof, such as the MIR29A sequence provided herein (e.g., SEQ ID NO: 1), or a functional equivalent of SEQ ID NO. 1, such as alleles, polymorphisms, or homologs thereof, or modified sequences that retain activity in reducing collagen type I and/or III production in LF cells or cultures of LF cells. A MIR29A reagent may be conspecific to a patient. As used herein “conspecific” refers to MIR29A reagents having sequences of mirRNA-29a of the same species as the patient. A MIR29A reagent may be delivered as a precursor or as one or both mature RNA products (for example and without limitation: hsa-miR-29a-5p and/or hsa-miR-29a-3p), which may be delivered in the same particles or different particles, e.g. when delivered by nanoparticle delivery. The RNA may be a nucleic acid analog, so long as the analog retains function as described herein, such as, for example and without limitation: 2′-O-methyl-substituted RNA, locked nucleic acid (LNA), bridged nucleic acid (BNA), morpholino, and peptide nucleic acid (PNA). The therapeutic MIR29A reagent may be single-stranded, as duplexed version of at least the mature miR-29a RNA may serve as inhibitors or decoys (see, e.g., Example 3).

The RNA or RNA analog, such as an RNAi reagent as described below, including microRNA reagents such as a MIR29A reagent, may be delivered to the LF, that is to cells of the LF (or “LF cells”) by any useful method. In one non-limiting example nanocarriers may be used to deliver the MIR29A reagent to LF cells. Examples of microcarrier delivery include, without limitation: lipid nanoparticles (see, e.g.; Hou, X., et al. Lipid nanoparticles for mRNA delivery. Nat Rev Mater 6, 1078-1094 (2021)), exosomes (see, e.g., Amiri, A., et al. Exosomes as bio-inspired nanocarriers for RNA delivery: preparation and applications. J Transl Med 20, 125 (2022)), liposomes (see, e.g., Xue H Y, et al. Lipid-Based Nanocarriers for RNA Delivery. Curr Pharm Des. 2015; 21(22):3140-7), polymer nanoparticles, dendrimers, or any other useful delivery vehicle, including targeted methods using antibodies or other target-specific binding reagent (See, generally, for example, Paunovska, K., Loughrey, D. & Dahlman, J. E. Drug delivery systems for RNA therapeutics. Nat Rev Genet 23, 265-280 (2022)).

Lipid nanoparticles are small particles that are endocytosed by cells, and their cargoes, such as an RNA, are released in the cell's lysosomes. Examples of lipid nanoparticles include the formulations used for the BioNTech/Pfizer BNT162b2 and Moderna mRNA-1273 vaccines for delivery of mRNAs and patisiran for delivery of siRNA. Compositions of lipid nanoparticles are broadly-described, and typically include: an ionizable lipid, a helper lipoid, a cholesterol, a PEGylated (polyethylene glycol-functionalized) lipid, and RNA in specific ratios. The choice of constituents for the LNPs.

Other transfection methods may be used to deliver a MIR29A reagent to LF cells. One alternative transfection method is by electroporation, by administering the MIR29A reagent, in any useful carrier or excipient, and, by using electrodes, e.g. a current is applied that increases cellular permeability to facilitate passage of a nucleic acid such as a MIR29A reagent into cells. Systems are commercially available that can be used for in vivo electroporation, such as the NEPA21 Super Electroporator equipped with in vivo, e.g., needle, electrodes (Nepa Gene Company, Ltd. Japan). Ultrasound (sonoporation) also may be used to deliver nucleic acids in vivo.

The MIR29A reagent may be delivered by a transfected or transduced gene, for example by viral-based, or plasmid-based delivery. An miRNA or shRNA RNAi reagent can be produced from a gene for expressing the miRNA or shRNA, transferred by any suitable means, such as by recombinant vector such as a recombinant Adeno-associated virus (AAV), Adenovirus (Ad), or retrovirus (e.g. lentiviral) vector, or by gene editing, such as by CRISPR-Cas or TALENS methods, as are broadly-known. See, e.g., Sung Y K, Kim S W. Recent advances in the development of gene delivery systems. Biomater Res. 2019 Mar. 12; 23:8 and Sharma D, et al., A review of the tortuous path of nonviral gene delivery and recent progress. Int J Biol Macromol. 2021 Jul. 31; 183:2055-2073, providing examples of viral and non-viral gene delivery methods.

A method of treating spinal stenosis in a patient, such as a human patient, is provided. The method comprises delivering to cells of a patient's LF (e.g. contacting cells of a patient's LF with) an amount of a MIR29A reagent effective to treat spinal stenosis in the patient, e.g., by reducing type I or type III collagen expression in LF cells of the patient, thereby reducing collagen deposition in the LF of the patient. The patient may have hypertrophic LF and the MIR29A reagent may be contacted with cells of the patient's hypertrophic LF. The LF cells may be contacted with a pharmaceutical composition comprising the MIR29A reagent, such as a lipid-based, exosome, or polymer-based nanoparticle comprising or associated with the MIR29A reagent, such as a lipid nanoparticle comprising the MIR29A reagent. The LF may be injected directly with the MIR29A-containing pharmaceutical composition, by epidural injection, optionally by image guidance, for example by X-ray or computer tomography-assisted (CT-assisted) injection. A single injection may be used, or a catheter may be placed to allow for multiple, or continuous infusion.

A method of treating LF hypertrophy in a patient, such as a human patient, is provided. The method comprises delivering to cells of a patient's LF (e.g., contacting cells of a patient's LF with) an amount of a MIR29A reagent effective to treat spinal stenosis in the patient, e.g., by reducing type I or type III collagen expression in LF cells of the patient, thereby reducing collagen deposition in the LF of the patient. The MIR29A reagent may be contacted with cells of the patient's hypertrophic LF. The LF cells may be contacted with a pharmaceutical composition comprising the MIR29A reagent, such as a lipid-based, exosome, or polymer-based nanoparticle comprising or associated with the MIR29A reagent, such as a lipid nanoparticle comprising the MIR29A reagent. The LF may be injected directly with the MIR29A-containing pharmaceutical composition, by epidural injection, optionally by image guidance, for example by X-ray or computer tomography-assisted (CT-assisted) injection. A single injection may be used, or a catheter may be placed to allow for multiple, or continuous infusion.

A method of reducing collagen, e.g., type I collagen and/or III collagen, production in a patient's, such as a human patient's, LF is provided. The method comprises delivering to cells of a patient's LF (e.g., contacting cells of a patient's LF with) an amount of an RNAi reagent, such as a MIR29A reagent effective to treat spinal stenosis in the patient, e.g., by reducing type I collagen or type III collagen expression in LF cells of the patient, thereby reducing collagen deposition in the LF of the patient. The patient may have hypertrophic LF and the MIR29A reagent may be contacted with cells of the patient's hypertrophic LF. The LF cells may be contacted with a pharmaceutical composition comprising the MIR29A reagent, such as a lipid-based, exosome, or polymer-based nanoparticle comprising or associated with the MIR29A reagent, such as a lipid nanoparticle comprising the MIR29A reagent. The LF may be injected directly with the MIR29A reagent-containing pharmaceutical composition, by epidural injection, optionally by image guidance, for example by X-ray or computer tomography-assisted (CT-assisted) injection. A single injection may be used, or a catheter may be placed to allow for multiple, or continuous infusion.

Also provided is a MIR29A reagent for use in treating spinal stenosis, LF hypertrophy, and/or to reduce type I and/or type III collagen expression in LF cells of a patient, such as a human patient, for example according to a method as described herein or in the preceding paragraphs.

In another aspect, Col I expression may be knocked down in a patient's LF tissue, e.g., to treat spinal stenosis in the patient. Knocking down Col I expression may be achieved by any effective method by delivery delivering effective amounts of a suitable reagent to cells of a patient's ligamentum flavum (LF), such as by use of antisense reagents or RNA interference reagents. Delivery of those reagents may be accomplished in the same manner as the MIR29A reagent described herein, e.g., using liposomes, LNPs, etc. Likewise, in another aspect, Col III expression may be knocked down in a patient's LF tissue, e.g., to treat spinal stenosis in the patient. Knocking down Col III expression may be achieved by any effective method by delivery delivering effective amounts of a suitable reagent to cells of a patient's ligamentum flavum (LF), such as by use of antisense reagents or RNA interference reagents. Delivery of those reagents may be accomplished in the same manner as the MIR29A reagent described herein, e.g., using liposomes, LNPs, etc.

Drug products, or pharmaceutical compositions comprising an active agent (e.g., drug), for example, a MIR29A reagent, or an active agent that decreases type I collagen expression or type III collagen expression, may be prepared by any method known in the pharmaceutical arts, for example, by bringing into association the active ingredient with the carrier(s) or excipient(s). As used herein, a “pharmaceutically acceptable excipient”, “carrier”, or “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable excipients include one or more of water, saline, phosphate buffered saline, dextrose, glycerol, ethanol and the like, as well as combinations thereof. In many cases, it may be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the active agent. In certain aspects, the active compound may be prepared with a carrier that will protect the compound against rapid release, such as a controlled release formulation, including implants, transdermal patches, and microencapsulated delivery systems. Biodegradable, biocompatible polymers can be used in delivery systems, such as ethylene vinyl acetate, polyanhydrides, polyglycolic acid, collagen, polyorthoesters, and polylactic acid. Many methods for the preparation of such formulations are broadly-known to those skilled in the art.

Additionally, active agent-containing compositions may be in a variety of forms. The preferred form depends on the intended mode of administration and therapeutic application, which will in turn dictate the types of carriers/excipients. Suitable forms include, but are not limited to, liquid, semi-solid and solid dosage forms.

Pharmaceutical formulations adapted for transdermal administration may be presented, for example and without limitation, as discrete patches intended to remain in intimate contact with the epidermis of the recipient for a prolonged period of time or electrodes for iontophoretic delivery.

Pharmaceutical formulations adapted for topical administration may be formulated, for example and without limitation, as ointments, creams, suspensions, lotions, powders, solutions, pastes, gels, sprays, aerosols, or oils.

Therapeutic compositions typically must be sterile and stable under the conditions of manufacture and storage. For example, sterile injectable solutions can be prepared by incorporating the active agent in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, typical methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

A “therapeutically effective amount” refers to an amount of a drug product or active agent effective, at dosages and for periods of time necessary, to achieve the desired therapeutic result. An “amount effective” for treatment of a condition is an amount of an active agent or dosage form, such as a single dose or multiple doses, effective to achieve a determinable end-point. The “amount effective” is preferably safe—at least to the extent the benefits of treatment outweighs the detriments, and/or the detriments are acceptable to one of ordinary skill and/or to an appropriate regulatory agency, such as the U.S. Food and Drug Administration. A therapeutically effective amount of an active agent may vary according to factors such as the disease state, age, sex, and weight of the individual, and the ability of the active agent to elicit a desired response in the individual. A “prophylactically effective amount” refers to an amount effective, at dosages and for periods of time necessary, to achieve a desired prophylactic result. Typically, since a prophylactic dose is used in subjects prior to or at an earlier stage of disease, the prophylactically effective amount may be less than the therapeutically effective amount.

Dosage regimens may be adjusted to provide the optimum desired response (e.g., a therapeutic or prophylactic response). For example, a single bolus may be administered, several divided doses may be administered over time, or the composition may be administered continuously or in a pulsed fashion with doses or partial doses being administered at regular intervals, for example, every 10, 15, 20, 30, 45, 60, 90, or 120 minutes, every 2 through 12 hours daily, or every other day, etc., be proportionally reduced or increased as indicated by the exigencies of the therapeutic situation. In some instances, it may be especially advantageous to formulate compositions, such as parenteral or inhaled compositions, in dosage unit form for ease of administration and uniformity of dosage. The specification for the dosage unit forms are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

By “target-specific” or reference to the ability of one compound to bind another target compound specifically, it is meant that the compound binds to the target compound to the exclusion of others in a given reaction system, e.g., in vitro, or in vivo, to acceptable tolerances, permitting a sufficiently specific diagnostic or therapeutic effect according to the standards of a person of skill in the art, a medical community, and/or a regulatory authority, such as the U.S. Food and Drug Agency (FDA), in aspects, in the context of administering a reagent as described herein to LF tissue in a patient.

A “gene” is a sequence of DNA or RNA which codes for a molecule, such as a protein or a functional RNA, such as an ncRNA that has a function. Nucleic acids are biopolymers, or small biomolecules, essential to all known forms of life. They are composed of nucleotides, which are monomers made of three components: a 5-carbon sugar, a phosphate group and a nitrogenous base. If the sugar is a simple ribose, the polymer is RNA; if the sugar is derived from deoxyribose, the polymer is DNA. DNA typically uses the nitrogenous bases guanine, thymine, adenine, and cytosine. RNA typically uses the nitrogenous bases guanine, uracil, adenine, and cytosine.

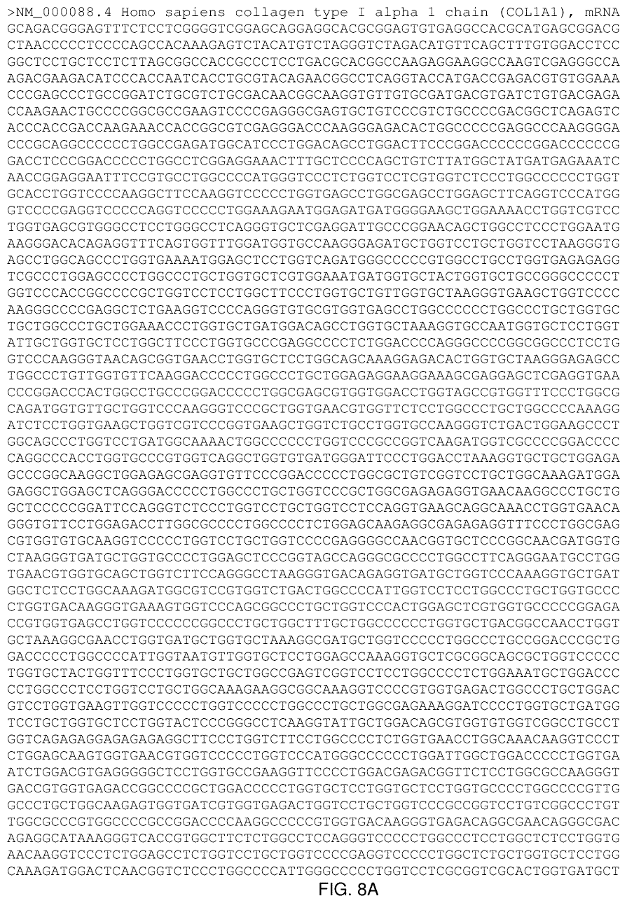

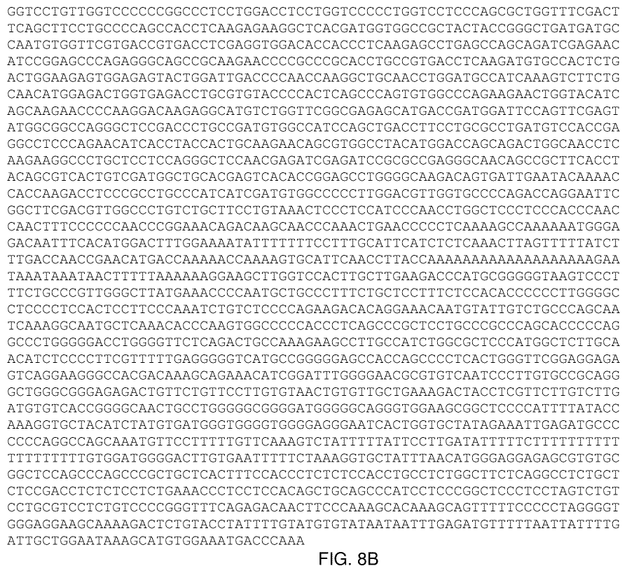

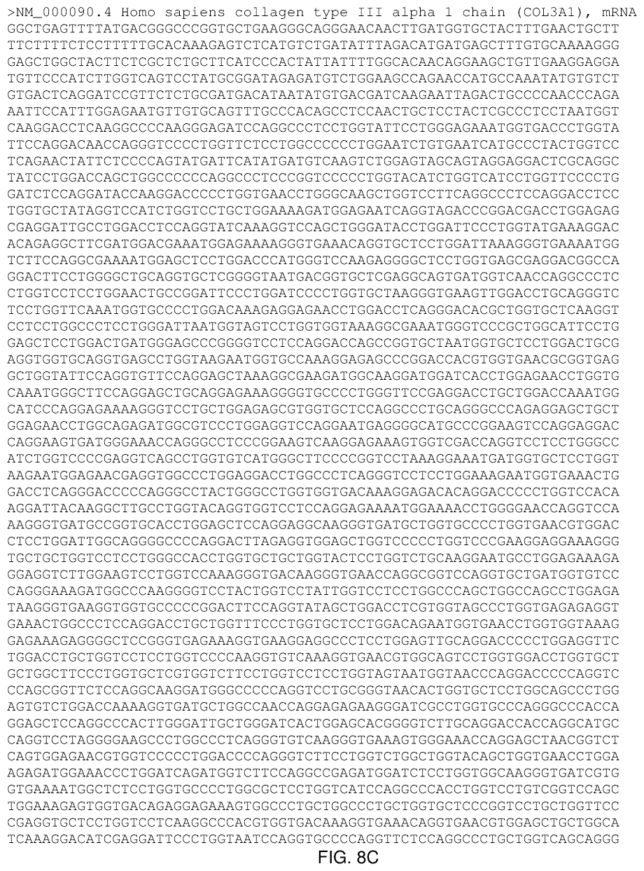

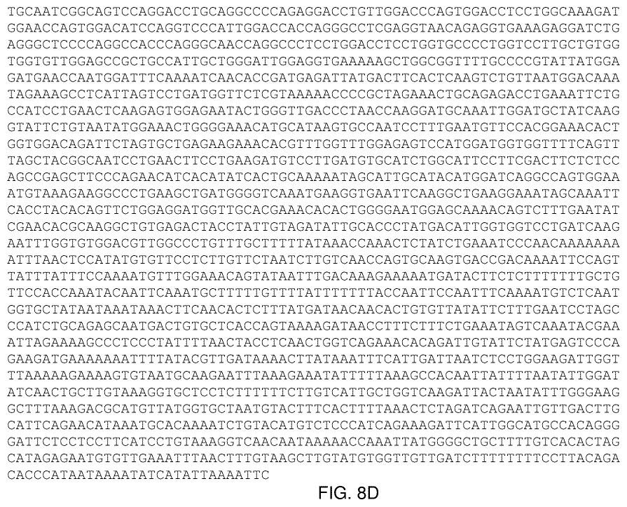

Complementary refers to the ability of polynucleotides (nucleic acids) to hybridize to one another, forming inter-strand base pairs. Base pairs are formed by hydrogen bonding between nucleotide units in antiparallel polynucleotide strands. Complementary polynucleotide strands can base pair (hybridize) in the Watson-Crick manner (e.g., A to T, A to U, C to G), or in any other manner that allows for the formation of duplexes. When using RNA as opposed to DNA, uracil rather than thymine is the base that is complementary to adenosine. Two sequences comprising complementary sequences can hybridize if they form duplexes under specified conditions, such as in water, saline (e.g., normal saline, or 0.9% w/v saline) or phosphate-buffered saline), or under other stringency conditions, such as, for example and without limitation, 0.1×SSC (saline sodium citrate) to 10×SSC, where 1×SSC is 0.15M NaCl and 0.015M sodium citrate in water. Hybridization of complementary sequences is dictated, e.g., by salt concentration and temperature, with the melting temperature (Tm) lowering with increased mismatches and increased stringency. Perfectly matched sequences are said to be fully complementary, or have 100% sequence identity (gaps are not counted and the measurement is in relation to the shorter of the two sequences). In one aspect, a sequence that “specifically hybridizes” to another sequence, does so in a hybridization solution containing 0.5M sodium phosphate buffer, pH 7.2, containing 7% SDS, 1 mM EDTA, and 100 mg/ml of salmon sperm DNA at 65° C. for 16 hours and washing twice at for twenty minutes in a washing solution containing 0.5×SSC and 0.1% SDS, or does so under conditions more stringent than 2×SSC at 65° C., for example, in 0.2×SSC at 55° C. A sequence that specifically hybridizes to another typically has at least 80%, 85%, 90%, 95%, or 99% sequence identity with the other sequence. It is noted that for purposes of the RNAi reagents described herein, the sequence of FIGS. 8A and 8B is a cDNA sequence (SEQ ID NO: 6), and for purposes of comparing sequence identity of an RNA sequence to that cDNA sequence, Ts and Us are interchangeable.

Gene expression is the process by which information from a gene is used in the synthesis of a functional gene product, e.g., a protein or functional RNA. Gene expression involves various steps, including transcription, post-transcriptional RNA modification, translation, and post-translational modification of a protein.

Transcription is the process by which the DNA gene sequence is transcribed into RNA. The steps include transcript initiation, transcript elongation, and transcript termination. The molecular machinery of transcription include but not limited to RNA polymerase, general transcription factors, enhancers, and promoter DNA, and RNA transcript. Transcription factors (TFs) are proteins that control the rate of transcription of genetic information from DNA to RNA, by binding to a specific DNA sequence (i.e., the promoter region). The function of TFs is to regulate genes in order to make sure that they are expressed in the right cell at the right time and in the right amount throughout the life of the cell and the organism. The promoter region of a gene is a region of DNA that initiates transcription of that particular gene. Promoters are located near the transcription start sites of genes, on the same strand, and often, but not exclusively, are upstream (towards the 5′ region of the sense strand) on the DNA. Promoters can be about 100-1000 base pairs long. Additional sequences and non-coding elements can affect transcription rates. If the cell has a nucleus (eukaryotes), the RNA is further processed. This includes polyadenylation, capping, and splicing. Polyadenylation is the addition of a poly(A) tail to a messenger RNA. The poly(A) tail consists of multiple adenosine monophosphates; in other words, it is a stretch of RNA that has only adenine bases. In eukaryotes, polyadenylation is part of the process that produces mature messenger RNA (mRNA) for translation. Capping refers to the process wherein the 5′ end of the pre-mRNA has a specially altered nucleotide. In eukaryotes, the 5′ cap (cap-0), found on the 5′ end of an mRNA molecule, consists of a guanine nucleotide connected to mRNA via an unusual 5′ to 5′ triphosphate linkage. During RNA splicing, pre-mRNA is edited. Specifically, during this process introns are removed and exons are joined together. The resultant product is known as mature mRNA. The RNA may remain in the nucleus or exit to the cytoplasm through the nuclear pore complex.

RNA levels in a cell, e.g., mRNA levels, can be controlled post-transcriptionally. Native mechanisms, including: endogenous gene silencing mechanisms, interference with translational mechanisms, interference with RNA splicing mechanisms, and destruction of duplexed RNA by RNAse H, or RNAse H-like activity. As is broadly-recognized by those of ordinary skill in the art, these endogenous mechanisms can be exploited to decrease or silence mRNA activity in a cell or organism in a sequence-specific, targeted manner. Antisense technology typically involves administration of a single-stranded antisense oligonucleotide (ASO) that is chemically-modified, e.g., as described herein, for bio-stability, and is administered in sufficient amounts to effectively penetrate the cell and bind in sufficient quantities to target mRNAs in cells. RNA interference (RNAi) harnesses an endogenous and catalytic gene silencing mechanism, which means that once, e.g., a microRNA, or double-stranded siRNA has been delivered into the cytosol, they are efficiently recognized and stably incorporated into the RNA-induced silencing complex (RiSC) to achieve prolonged gene silencing. Both antisense technologies and RNAi have their strengths and weaknesses, either may be used effectively to knock-down or silence expression of a gene or gene product, such as a collagen, such as a Type I collagen (see, e.g., Watts, J. K., et al. Gene silencing by siRNAs and antisense oligonucleotides in the laboratory and the clinic (2012) 226(2):365-379 and Nath, R K, et al., Protective Effect of Type I Collagen Antisense Oligonucleotides on Bleomycin Induced Pulmonary Fibrosis, The Open Conference Proceeding Journal 2010 1:141-49, using antisense reagent AS61-ODN-5′ ACTGTCTTCTTGGCCATGCG-3′ (SEQ ID NO: 5), which significantly reduced mRNA and protein expression of type 1 collagen in bleomycin induced pulmonary fibrosis model in mice) and Type II collagen.

The terms “iRNA,” “RNAi reagent,” and “RNA interference agent” as used interchangeably herein, refer to an agent that contains RNA nucleotides, and which mediates the targeted cleavage of an RNA transcript via an RNA-induced silencing complex (RISC) pathway. iRNA directs the sequence-specific degradation of mRNA through a process known as RNA interference (RNAi). The iRNA modulates, e.g., knocks down or silences, the expression of type 1 collagen RNA in a cell, e.g., a cell within a subject, such as a mammalian subject. Non-limiting examples of RNAi reagents include siRNA, microRNA, and shRNA.

In one aspect, an RNAi reagent includes a single stranded RNAi that interacts with a target RNA sequence, e.g., a type I collagen RNA sequence, to direct the cleavage of the target RNA. Without wishing to be bound by theory it is believed that long double stranded RNA introduced into cells is broken down into double stranded short interfering RNAs (siRNAs) comprising a sense strand and an antisense strand by a Type III endonuclease known as Dicer. Dicer, a ribonuclease-III-like enzyme, processes these dsRNA into 19-23 base pair short interfering RNAs with characteristic two base 3′ overhangs. These siRNAs are then incorporated into an RNA-induced silencing complex (RISC) where one or more helicases unwind the siRNA duplex, enabling the complementary antisense strand to guide target recognition. Upon binding to the appropriate target mRNA, one or more endonucleases within the RISC cleave the target to induce silencing. Thus, in one aspect the invention relates to a single stranded RNA (ssRNA) (the antisense strand of an siRNA duplex) generated within a cell and which promotes the formation of a RISC complex to effect silencing of the target gene. Accordingly, the term “siRNA” is also used herein to refer to an interfering RNA (iRNA).

In another aspect, the RNAi reagent may be a single-stranded RNA that is introduced into a cell or organism to inhibit a target mRNA. Single-stranded RNAi reagents bind to the RISC endonuclease, Argonaute 2, which then cleaves the target mRNA. The single-stranded siRNAs are generally 15-30 nucleotides and are chemically modified. The design and testing of single-stranded RNAs are described in U.S. Pat. No. 8,101,348 and in Lima et al., (2012) Cell 150:883-894. Any of the RNAi reagents described herein may be used as a single-stranded siRNA as described herein or as chemically modified by the methods described in Lima et al.

In another aspect, an “iRNA” or RNAi reagent” for use in the compositions and methods described herein is a double stranded RNA and can be referred to herein as a “double stranded RNAi reagent,” “double stranded RNA (dsRNA) molecule,” “dsRNA agent,” or “dsRNA”. The term “dsRNA”, refers to a complex of ribonucleic acid molecules, having a duplex structure comprising two anti-parallel and substantially complementary nucleic acid strands, referred to as having “sense” and “antisense” orientations with respect to a target RNA, e.g., a type 1 collagen RNA. In some aspects, a double stranded RNA (dsRNA) triggers the degradation of a target RNA, e.g., an mRNA, through a post-transcriptional gene-silencing mechanism referred to herein as RNA interference or RNAi.

The majority of nucleotides of each strand of a dsRNA molecule may be ribonucleotides, but as described in detail herein, each or both strands can also include nucleotide analogs, where one or more non-ribonucleotides, e.g., a deoxyribonucleotide and/or a modified nucleotide. In addition, as used in this specification, an “RNAi reagent” may include ribonucleotides with chemical modifications; an RNAi reagent may include substantial modifications at multiple nucleotides. As used herein, the term “modified nucleotide” refers to a nucleotide having, independently, a modified sugar moiety, a modified inter-nucleotide linkage, and/or modified nucleobase. Thus, the term modified nucleotide encompasses substitutions, additions or removal of, e.g., a functional group or atom, to inter-nucleoside linkages, sugar moieties, or nucleobases. The modifications suitable for use in the agents described herein include all types of modifications disclosed herein or known in the art. Any such modifications, as used in a siRNA type molecule, are encompassed by “RNAi reagent” or “RNAi reagent” for the purposes of this disclosure.

The duplex region may be of any length that permits specific degradation of a desired target RNA through a RISC pathway, and may range from about 9 to 36 base pairs in length, e.g., about 15-30 base pairs in length, for example, about 9, 10, 11, 12, 13, 14, 15, 16, 17, 18, 19, 20, 21, 22, 23, 24, 25, 26, 27, 28, 29, 30, 31, 32, 33, 34, 35, or 36 base pairs in length, such as about 15-30, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-25, 20-24, 20-23, 20-22, 20-21, 21-30, 21-29, 21-28, 21-27, 21-26, 21-25, 21-24, 21-23, or 21-22 base pairs in length. Ranges and lengths intermediate to the above recited ranges and lengths are also contemplated to be part of the invention.

The two strands forming the duplex structure may be different portions of one larger RNA molecule, or they may be separate RNA molecules. Where the two strands are part of one larger molecule, and therefore are connected by an uninterrupted chain of nucleotides between the 3′-end of one strand and the 5′-end of the respective other strand forming the duplex structure, the connecting RNA chain is referred to as a “hairpin loop.” A hairpin loop can comprise at least one unpaired nucleotide. In some aspects, the hairpin loop can comprise at least 2, at least 3, at least 4, at least 5, at least 6, at least 7, at least 8, at least 9, at least 10, at least 20, at least 23, or more unpaired nucleotides. In some aspects, the hairpin loop can be 10 or fewer nucleotides. In some aspects, the hairpin loop can be 8 or fewer unpaired nucleotides. In some aspects, the hairpin loop can be 4-10 unpaired nucleotides. In some aspects, the hairpin loop can be 4-8 nucleotides.

Where the two substantially complementary strands of a dsRNA are comprised by separate RNA molecules, those molecules need not, but can be covalently connected. Where the two strands are connected covalently by means other than an uninterrupted chain of nucleotides between the 3′-end of one strand and the 5′-end of the respective other strand forming the duplex structure, the connecting structure is referred to as a “linker.” The RNA strands may have the same or a different number of nucleotides. The maximum number of base pairs is the number of nucleotides in the shortest strand of the dsRNA minus any overhangs that are present in the duplex. In addition to the duplex structure, an RNAi may comprise one or more nucleotide overhangs.

In one aspect, an RNAi reagent is a dsRNA, each strand of which comprises 19-23 nucleotides, that interacts with a target RNA sequence, e.g., a type I collagen RNA, without wishing to be bound by theory, long double stranded RNA introduced into cells is broken down into siRNA by a Type III endonuclease known as Dicer. Dicer, a ribonuclease-III-like enzyme, processes the dsRNA into 19-23 base pair short interfering RNAs with characteristic two base 3′ overhangs. The siRNAs are then incorporated into an RNA-induced silencing complex (RISC) where one or more helicases unwind the siRNA duplex, enabling the complementary antisense strand to guide target recognition. Upon binding to the appropriate target RNA, one or more endonucleases within the RISC cleave the target to induce silencing. In one aspect, an RNAi reagent is a dsRNA of 24-30 nucleotides that interacts with a target RNA sequence, e.g., a type 1 collagen RNA sequence, to direct the cleavage of the target RNA.

As used herein, the term “nucleotide overhang” refers to at least one unpaired nucleotide that protrudes from the duplex structure of an iRNA, e.g., a dsRNA. For example, when a 3′-end of one strand of a dsRNA extends beyond the 5′-end of the other strand, or vice versa, there is a nucleotide overhang. A dsRNA can comprise an overhang of at least one nucleotide; alternatively, the overhang can comprise at least two nucleotides, at least three nucleotides, at least four nucleotides, at least five nucleotides or more. A nucleotide overhang can comprise or consist of a nucleotide/nucleoside analog, including a deoxynucleotide/nucleoside. The overhang(s) can be on the sense strand, the antisense strand or any combination thereof. Furthermore, the nucleotide(s) of an overhang can be present on the 5′-end, 3′-end or both ends of either an antisense or sense strand of a dsRNA.

In one aspect of the dsRNA, at least one strand comprises a 3′ overhang of at least 1 nucleotide. In another aspect, at least one strand comprises a 3′ overhang of at least 2 nucleotides, e.g., 2, 3, 4, 5, 6, 7, 9, 10, 11, 12, 13, 14, or 15 nucleotides. In other aspects, at least one strand of the RNAi reagent comprises a 5′ overhang of at least 1 nucleotide. In certain aspects, at least one strand comprises a 5′ overhang of at least 2 nucleotides, e.g., 2, 3, 4, 5, 6, 7, 9, 10, 11, 12, 13, 14, or 15 nucleotides. In still other aspects, both the 3′ and the 5′ end of one strand of the RNAi reagent comprise an overhang of at least one nucleotide.

In one aspect, the antisense strand of a dsRNA has a 1-10 nucleotide, e.g., a 1, 2, 3, 4, 5, 6, 7, 8, 9, or 10 nucleotides, overhang at the 3′-end and/or the 5′-end. In one aspect, the sense strand of a dsRNA has a 1-10 nucleotide, e.g., a 1, 2, 3, 4, 5, 6, 7, 8, 9, or 10 nucleotides, overhang at the 3′-end and/or the 5′-end. In certain aspects, the overhang on the sense strand or the antisense strand, or both, can include extended lengths longer than 10 nucleotides, e.g., 1-30 nucleotides, 2-30 nucleotides, 10-30 nucleotides, or 10-15 nucleotides in length. In certain aspects, an extended overhang is on the sense strand of the duplex. In certain aspects, an extended overhang is present on the 3′ end of the sense strand of the duplex. In certain aspects, an extended overhang is present on the 5′ end of the sense strand of the duplex. In certain aspects, an extended overhang is on the antisense strand of the duplex. In certain aspects, an extended overhang is present on the 3′ end of the antisense strand of the duplex. In certain aspects, an extended overhang is present on the 5′ end of the antisense strand of the duplex. In another aspect, one or more of the nucleotides in the overhang is replaced with a nucleoside thiophosphate.

The terms “blunt” or “blunt ended” as used herein in reference to a dsRNA mean that there are no unpaired nucleotides or nucleotide analogs at a given terminal end of a dsRNA, i.e., no nucleotide overhang. One or both ends of a dsRNA can be blunt.

Where both ends of a dsRNA are blunt, the dsRNA is said to be blunt ended. To be clear, a “blunt ended” dsRNA is a dsRNA that is blunt at both ends, i.e., no nucleotide overhang at either end of the molecule. Most often such a molecule will be double stranded over its entire length.

The term “antisense strand” or “guide strand” refers to the strand of an iRNA, e.g., a dsRNA, which includes a region that is substantially complementary to a target sequence, e.g., a type I collagen RNA. As used herein, the term “region of complementarity” refers to the region on the antisense strand that is substantially complementary to a sequence, for example, a target sequence, e.g., a type I collagen RNA sequence, e.g., as described herein. Where the region of complementarity is not fully complementary to the target sequence, the mismatches can be in the internal or terminal regions of the molecule. Generally, the most tolerated mismatches are in the terminal regions, e.g., within 5, 4, 3, or 2 nucleotides of the 5′- and/or 3′-terminus of the iRNA.

In one non-limiting example, a collagen is exemplified by the human COL1A1 collagen type I alpha 1 chain (Gene ID 1277; e.g., NCBI Reference Sequence: NM_000088.4, as depicted in FIGS. 8A and 8B). In another non-limiting example, a collagen is exemplified by the human COL3A1 collagen type III alpha 1 chain (Gene ID: 1281; e.g., NCBI Reference Sequence: NM_000090.4, as depicted in FIGS. 8C and 8D (SEQ ID NO: 7)). Using these sequences, a person of ordinary skill can determine suitable RNAi reagent sequences, e.g., by use of any suitable computer-based method, algorithm, or guidelines for RNAi selection and development, as are broadly-available. Further siRNA, shRNA, and other RNAi reagents, as well as ASO reagents can be obtained commercially, e.g. from ThermoFisher Scientific, among many others.

The term “sense strand” or “passenger strand” as used herein, refers to the strand of an iRNA that includes a region that is substantially complementary to a region of the antisense strand as that term is defined herein.

As used herein, the term “cleavage region” refers to a region that is located immediately adjacent to the cleavage site. The cleavage site is the site on the target at which cleavage occurs. In some aspects, the cleavage region comprises three bases on either end of, and immediately adjacent to, the cleavage site. In some aspects, the cleavage region comprises two bases on either end of, and immediately adjacent to, the cleavage site. In some aspects, the cleavage site specifically occurs at the site bound by nucleotides 10 and 11 of the antisense strand, and the cleavage region comprises nucleotides 11, 12, and 13.

Complementary sequences within an iRNA, e.g., within a dsRNA as described herein, include base-pairing of the oligonucleotide or polynucleotide comprising a first nucleotide sequence to an oligonucleotide or polynucleotide comprising a second nucleotide sequence over the entire length of one or both nucleotide sequences. Such sequences can be referred to as “fully complementary” with respect to each other herein. However, where a first sequence is referred to as “substantially complementary” with respect to a second sequence herein, the two sequences can be fully complementary, or they can form one or more, but generally not more than 5, 4, 3, or 2 mismatched base pairs upon hybridization for a duplex up to 30 base pairs, while retaining the ability to hybridize under the conditions most relevant to their ultimate application, e.g., inhibition of gene expression via a RISC pathway. However, where two oligonucleotides are designed to form, upon hybridization, one or more single stranded overhangs, such overhangs shall not be regarded as mismatches with regard to the determination of complementarity. For example, a dsRNA comprising one oligonucleotide 21 nucleotides in length and another oligonucleotide 23 nucleotides in length, wherein the longer oligonucleotide comprises a sequence of 21 nucleotides that is fully complementary to the shorter oligonucleotide, can yet be referred to as “fully complementary” for the purposes described herein.

“Complementary” sequences, as used herein, can also include, or be formed entirely from, non-Watson-Crick base pairs and/or base pairs formed from non-natural and modified nucleotides, in so far as the above requirements with respect to their ability to hybridize are fulfilled. Such non-Watson-Crick base pairs include, but are not limited to, G:U Wobble or Hoogstein base pairing.

The terms “complementary,” “fully complementary,” and “substantially complementary” herein can be used with respect to the base matching between the sense strand and the antisense strand of a dsRNA, or between the antisense strand of an RNAi reagent and a target sequence, as will be understood from the context of their use.

As used herein, a polynucleotide that is “substantially complementary to at least part of a messenger RNA (mRNA)” refers to a polynucleotide that is substantially complementary to a contiguous portion of the mRNA of interest (e.g., a type I collagen RNA or a type III collagen RNA).

Accordingly, in some aspects, the antisense strand polynucleotides disclosed herein are fully complementary to the target collagen, e.g. type I collagen RNA sequence or type III collagen RNA sequence. In other aspects, the antisense strand polynucleotides disclosed herein are substantially complementary to the target collagen, e.g., type I collagen RNA sequence or type III collagen RNA sequence and comprise a contiguous nucleotide sequence which has at least about 80% sequence identity to the nucleotide sequence of FIGS. 8A and 8B or the nucleotide sequence of FIGS. 8C and 8D), or a fragment thereof, such as about 85%, about 86%, about 87%, about 88%, about 89%, about 90%, about 91%, about 92%, about 93%, about 94%, about 95%, about 96%, about 97%, about 98%, or about 99% complementary.

It is understood that the sequence of the type I collagen RNA or type III collagen RNA must be sufficiently complementary to the antisense strand of the RNAi reagent for the agent to be used in the indicated patient, e.g. human, mammalian, or vertebrate species.

The term “inhibiting,” as used herein, is used interchangeably with “reducing,” “silencing,” “downregulating,” “suppressing,” “knocking down,” and other similar terms, and includes any level of inhibition.

The phrases “knocking down or silencing of type I collagen RNA” or “knocking down or silencing of type III collagen RNA” as used herein, includes inhibition of expression of any type I collagen or type III collagen gene (such as, e.g., a mouse type I collagen gene, a rat type I collagen gene, a monkey type I collagen gene, or a human type I collagen gene, a mouse type III collagen gene, a rat type III collagen gene, a monkey type I collagen gene, or a human type III collagen gene) as well as variants or mutants of an type I collagen gene or type III collagen gene, in its production of type I collagen RNA or type III collagen RNA, affecting the stability of type I collagen RNA or type III collagen RNA, such as by antisense or RNAi technologies. “Knocking down or silencing of type I collagen RNA” includes any level of inhibition of a type I collagen RNA, e.g., at least partial suppression of the expression of a type I collagen RNA, such as an inhibition by at least about 20%. In certain aspects, inhibition is by at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. “Knocking down or silencing of type III collagen RNA” includes any level of inhibition of a type III collagen RNA, e.g., at least partial suppression of the expression of a type III collagen RNA, such as an inhibition by at least about 20%. In certain aspects, inhibition is by at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%.

The expression of a type I collagen RNA or a type III collagen RNA may be assessed based on the level of any variable associated with type I collagen RNA or type III collagen RNA expression, e.g., type I collagen RNA or type III collagen RNA level. The expression of a type I collagen RNA or a type III collagen RNA may also be assessed indirectly based on assay of physiological markers associated with decreased expression of the type I collagen RNA or the type III collagen RNA in a patient or a tumor cell.

In one aspect, at least partial suppression of the expression of a type I collagen RNA or a type III collagen RNA, is assessed by a reduction of the amount of type I collagen RNA or a type III collagen RNA that can be isolated from or detected in a cell or group of cells, e.g., in a tumor cell. As such, in aspects, type I collagen levels or type III collagen RNA levels are determined from a biopsy, or from a normal tissue sample obtained from a patient. A reduction of the amount of type I collagen RNA or type III collagen RNA in a cell or tissue in which a type I collagen gene or a type III collagen gene is transcribed and which has been treated such that the expression of a type I collagen RNA or a type III collagen RNA is inhibited, may be determined as compared to a second cell or tissue substantially identical to the first cell or tissue but which has not been so treated (control cells). The degree of inhibition may be expressed in terms of:

((mRNA in control cells)−(mRNA in treated cells))/((mRNA in control cells))×100%).

The phrase “contacting a cell with an RNAi reagent,” such as a dsRNA or miRNA, as used herein, includes contacting a cell by any possible means. Contacting a cell with an RNAi reagent includes contacting a cell in vitro with the iRNA or contacting a cell in vivo with the iRNA. The contacting may be done directly or indirectly. Thus, for example, the RNAi reagent may be put into physical contact with the cell by the individual performing the method, or alternatively, the RNAi reagent may be put into a situation that will permit or cause it to subsequently come into contact with the cell. Further, an shRNA RNAi reagent or pri-miRNA RNAi reagent can be produced from a gene for expressing an shRNA or pri-miRNA, transferred by any suitable means, such as by recombinant vector such as a recombinant Adeno-associated virus (AAV) or retrovirus vector, or by gene editing, such as by CRISPR-Cas or TALENS methods, as are broadly-known. These technologies are broadly-known by those of ordinary skill and resources, such as suitable vectors and production systems are broadly-available, including from commercial sources.

Contacting a cell in vitro may be done, for example, by incubating the cell with the RNAi reagent. Contacting a cell in vivo may be done, for example, by injecting the RNAi reagent into or near the tissue where the cell is located, such as the LF, or by injecting the RNAi reagent into another area, e.g., the bloodstream or the subcutaneous space, such that the agent will subsequently reach the tissue where the cell to be contacted is located. For example, the RNAi reagent may contain and/or be coupled to a ligand that directs the RNAi reagent to a site of interest. Combinations of in vitro and in vivo methods of contacting are also possible. For example, a cell may also be contacted in vitro with an RNAi reagent and subsequently transplanted into a subject.

In one aspect, contacting a cell with an RNAi reagent includes “introducing” or “delivering the RNAi reagent into the cell” by facilitating or effecting uptake or absorption into the cell. Absorption or uptake of an RNAi reagent can occur through unaided diffusive or active cellular processes, or by use of auxiliary agents or devices. Introducing an RNAi reagent into a cell may be in vitro and/or in vivo. For example, for in vivo introduction, RNAi reagent can be injected into a tissue site or administered systemically. In vitro introduction into a cell includes methods known in the art such as electroporation, lipofection, including delivery via LNPs. Further approaches are described herein below and/or are known in the art.

As used herein, and further to the discussion above regarding RNAi reagents, “agent” or “RNAi reagent,” when used in the context of an antisense, siRNA, shRNA, miRNA, pri-miRNA, or ribozyme, or other single-stranded or double-stranded RNA interfering nucleic acids, refers not only to RNA structures, but effective nucleic acid analog structures. In antisense and RNAi technologies, use of RNA can pose significant delivery issues due to the lability of RNA molecules. As such, RNA may be chemically-modified to produce nucleic acid analogs, not only to enhance stability of the nucleic acid molecules, but often resulting in increased binding affinity, and with reduced toxicity. Such modifications are broadly-known to those of ordinary skill in the art, and are available commercially (see, e.g., Corey, D. R., Chemical modification: the key to clinical application of RNA interference? (2007) J Clin Invest. 117(12):3615-3622, also describing RNAi, and United States Patent Application Publication No. 2017/0081667, incorporated herein by reference for its technical disclosure). Non-limiting examples of modifications to the nucleic acid structure in nucleic acid analogs include: modifications to the phosphate linkage, such as phosphoramidates or phosphorothioates; sugar modification, such as 2′-O, 4′-C methylene bridged, locked nucleic acid (LNA), 2′-methoxy, 2′-O-methoxyethyl (MOE), 2′-fluoro, S-constrained-ethyl (cEt), and tricyclo-DNA (tc-DNA); and non-ribose structures, such as phosphorodiamidate morpholino (PMO) and peptide-nucleic acids (PNA).

In addition to those MIR29A, type I collagen-active RNAi reagents or type III collagen-active RNAi reagents described herein, antisense agents (ASOs), other RNAi reagents, ribozyme agents, and other nucleic acid-based methods of reducing gene expression, can be designed and tested based on known sequences of type I or type III collagen RNAs and gene structure (exemplary sequence provided herein). Based on the present disclosure, one of ordinary skill can design, and/or produce an active agent capable of knocking down type I collagen and/or type III collagen expression. Of note, a number of publications describe algorithms for generating candidate RNAi reagent sequences, and publicly-available software can be used to implement those algorithms. As such, typically, one only needs to enter an mRNA sequence into a calculator to produce candidate RNAi reagents.

RNAi reagents may have 100% sequence identity with a portion or fragment of the type I collagen or type III collagen sequences provided herein, or a sequence complementary thereto, or may include one or more additional nucleobases at their 3′ or 5′ end, or may include one or more substitutions that do not substantially interfere with the activity of the RNAi reagent in knocking down or silencing type I collagen or type III collagen expression. Alleles, mutations, or other variants or polymorphisms (e.g., single-nucleotide polymorphisms, SNPs) of type I collagen or type III collagen sequences are possible, and as such effective agents, such as RNAi and antisense agents may be substituted to accommodate those variants. Further, some sequence mismatches in RNAi reagents are not only tolerated, but may be beneficial (see, e.g., Wu, H., et al. “Improved siRNA/shRNA Functionality by Mismatched Duplex” PLoS One. 2011; 6(12): e28580). As such, sequences having up to 90% or 95% (two or one mismatches, respectively) sequence identity with the sequence provided in FIGS. 8A and 8B or the sequence provided in FIGS. 8C and 8D are expected, in many circumstances, to be effective RNAi reagents.

Therefore, according to one aspect, provided herein is a method of treating spinal stenosis in a patient, comprising knocking down or silencing type I collagen expression or activity to a level effective to treat spinal stenosis in the patient. Type I collagen expression can be knocked down or silenced, e.g., by use of antisense nucleic acids, or by use of RNAi reagents. In one aspect expression of the type I collagen gene is silenced by administration of an RNAi reagent to the patient, such as a siRNA, as described above and which are commercially available.

In aspects, by knocking down or silencing type I collagen RNA expression or activity, it is meant any action that results in lower activity of type I collagen in a cell or patient—typically by use of a therapeutic agent. Useful therapeutic agents include, without limitation, antisense or RNAi compositions.

Therefore, according to another aspect, provided herein is a method of treating spinal stenosis in a patient, comprising knocking down or silencing type III collagen expression or activity to a level effective to treat spinal stenosis in the patient. Type III collagen expression can be knocked down or silenced, e.g., by use of antisense nucleic acids, or by use of RNAi reagents. In one aspect expression of the type III collagen gene is silenced by administration of an RNAi reagent to the patient, such as a siRNA, as described above and which are commercially available.

In aspects, by knocking down or silencing type III collagen RNA expression or activity, it is meant any action that results in lower activity of type III collagen in a cell or patient—typically by use of a therapeutic agent. Useful therapeutic agents include, without limitation, antisense or RNAi compositions.

U.S. Pat. No. 7,737,265 and International Patent Publication No. WO 2016/209862, each of which is incorporated herein by reference for its technical disclosure to the extent it is consistent with the present disclosure, are examples of the many publications disclosing further details regarding iRNA technology and RNAi reagents, the disclosure of which is broadly applicable to methods of making and using agents for use in knocking down or silencing type I collagen expression and/or type III collagen expression, as described herein. Disclosed in WO/2016/209862 are details relating to iRNA structure, definition of required sequences and agent size, definitions and descriptions of target sequences, methods of making iRNAs, variations or modifications in iRNA structures, such as nucleic acid analogs or mimetics, methods of modification of iRNAs such as ligand-modified iRNAs, including polysaccharide-modified or polypeptide-modified iRNAs and linkers that can be useful in targeting the iRNA, pharmaceutical compositions for delivery of iRNAs, delivery methods and delivery routes for iRNAs, including liposome or micellar delivery systems, and methods of determining whether iRNAs are effective. One of ordinary skill in the art can identify and optimize type I collagen and/or type III collagen RNAi reagents based on available knowledge and resources. Further disclosure of how to identify, make, or use type I collagen and/or type III collagen RNAi reagents is unnecessary.

Viral delivery of miRNA and shRNA (iRNA) may be accomplished using any of a number of commercially-available and well-characterized systems for producing recombinant viral transducing particles. Non-limiting examples of suitable viral vectors include lentiviral or retroviral, adenoviral, adeno-associated virus, or herpesvirus vector systems. By “expression” or “gene expression,” it is meant the overall flow of information from a gene (without limitation, a functional genetic unit for producing a gene product, such as a functional RNA (e.g. miRNA or shRNA, among others) or a protein in a cell, or other expression system encoded on a nucleic acid and comprising: a transcriptional control sequence, such as a promoter and other cis-acting elements, such as transcriptional response elements (TREs) and/or enhancers; an expressed sequence that typically encodes a protein (referred to as an open-reading frame or ORF) or functional/structural RNA, and a polyadenylation sequence), to produce a gene product (typically a protein, optionally post-translationally modified or a functional/structural RNA such as a MIR29A reagent). By “expression of genes under transcriptional control of,” or alternately “subject to control by,” a designated sequence such as TRE or transcription control element, it is meant gene expression from a gene containing the designated sequence operably linked (functionally attached, typically in cis) to the gene. A gene that is “under transcriptional control” of a TRE or transcription control element, is a gene that is transcribed at detectably different levels in the presence of a transcription factor. The designated sequence may be all or part of the transcriptional control elements (without limitation, promoters, TREs, enhancers and response elements), and may wholly or partially regulate and/or affect transcription of a gene. A “gene for expression of” a stated gene product is a gene capable of expressing that stated gene product when placed in a suitable environment—that is, for example, when transformed, transfected, transduced, etc. into a cell, and subjected to suitable conditions for expression. In the case of a constitutive promoter “suitable conditions” means that the gene typically need only be introduced into a host cell. In the case of an inducible promoter, “suitable conditions” means when factors that regulate transcription, such as DNA-binding proteins, are present or absent—for example an amount of the respective inducer is available to the expression system (e.g., cell), or factors causing suppression of a gene are unavailable or displaced—effective to cause expression of the gene.

The nucleic acids are optionally, and preferably in many instances, recombinant, packaged viral genomes (nucleic acid that can be packaged into a viral particle), such that the nucleic acid is part of a transduction particle by which a cell can be transfected, as is broadly-known, for example as described in detail below regarding rAAV technologies.

AAV (adeno-associated virus), is a virus belonging to the genus Dependoparvovirus, and family Parvoviridae. The virus is a small replication-defective, non-enveloped virus. AAV is not currently known to cause any disease by itself. AAV requires a helper virus, such as adenovirus or herpes simplex virus, to facilitate productive infection and replication. In the absence of helper virus, AAVs establish a latent infection within the cell, either by site-specific integration into the host genome or by persisting in episomal forms. Gene therapy vectors using AAV can infect both dividing and quiescent cells. Furthermore, AAV serotypes have different tropism and can infect cells of multiple diverse tissue types. While eleven serotypes of AAV have been identified to date, AAV2 was among the first to be identified and has been consistently used for the generation of recombinant AAV vectors.

The AAV virion shell is approximately 25 nm in diameter and encapsulates a single-stranded DNA genome that consists of two large open reading frames (ORFs) flanked by inverted terminal repeats (ITR). The ITRs are the only cis-acting elements required for genome replication and packaging. In wild-type AAV, the left ORF encodes four replication proteins responsible for site-specific integration, nicking, and helicase activity, as well as regulation of promoters within the AAV genome. AAV possesses a 4.7 kb genome, and as such, efficient packaging of recombinant AAV (rAAV) vectors can be performed with constructs ranging from 4.1 kb to 4.9 kb in size (See, e.g., Samulski, R J, et al., AAV-Mediated Gene Therapy for Research and Therapeutic Purposes, Annu. Rev. Virol. 2014. 1:427-51).

Helper-free production of the rAAV requires transfection of the following components into host cells, typically 293 cells (HEK293 cells), which are broadly available, or similar cell lines: (1) an rAAV vector containing the transgene expression cassette flanked by the two ITRs, (2) expression of Rep and Cap proteins, typically provided by a helper plasmid in trans, and (3) adenovirus genes encoding E1, E2A, E4, and virus-associated RNA, also provided, at least in part by another helper plasmid in trans (293 cells produce the Ad E1 gene in trans). Rep and Cap proteins, which are necessary for viral packaging, are replication proteins and capsid proteins, respectively. Rep proteins consist of rep 78, 68, 52 and 40. They specifically are involved with the replication of AAV. Cap proteins are comprised of three proteins, VP1, VP2 and VP3, with molecular weight of 87, 72 and 62 kDa, respectively. These capsid proteins assemble into a near-spherical protein shell of 60 subunits. Helper-free AAV packaging systems are broadly available, for example from Clontech of Mountain View, California, from Cell Biolabs, Inc. of San Diego, CA, and see, e.g., U.S. Pat. Nos. 6,093,570, 6,458,587, 6,951,758, and 7,439,065. In scAAV (self-complementary AAV), the right ITR contains a deletion of D-sequence (the packaging signal) and a terminal resolution site mutation (Δtrs), which prevent Rep-mediated nicking and force packaging of dimer or self-complementary genomes (see FIG. 8). Making dsAAV from scAAV vector renders much improved transduction both in vitro and in vivo (see, e.g., pscAAV-MCS Expression vector, Product Data Sheet, Cell Biolabs, Inc., San Diego, California (2015)).

Preparation of rAAV transducing particles, such as scAAV transducing particles is routine. Since the transfection method is often considered unsuitable for large-scale production, the infection of cell lines stably expressing Rep and Cap with adenovirus carrying a vector genome has afforded the ability to scale-up. Another option includes infection of proviral cell lines with adenovirus or herpes simplex virus vector carrying an AAV Rep and Cap expression cassette. These methods still require the complete elimination of adenovirus (or herpesvirus) during the production process. However, in baculovirus expression vector systems for rAAV vector production in insect SF9 cells, the components of AAV production, including Rep and Cap proteins, as well as vector genomes are provided by separate recombinant baculoviruses. Ayuso, E., “Manufacturing of recombinant adeno-associated viral vectors: new technologies are welcome”, Molecular Therapy—Methods & Clinical Development (2016) 3, 15049; doi:10.1038/mtm.2015.49, and Merten, O-W, et al., describe numerous robust current rAAV production methods, though commercial scale-up and validation needs improvement. High viral titers (˜10¹²-10¹³ vp/mL) may be required for certain uses described herein. Protocols are available in the literature for concentration and purification of AAV vectors, allowing production of virus at these high concentrations (see, e.g., Gray S J, et al. (2011) Production of recombinant adeno-associated viral vectors and use in in vitro and in vivo administration. Curr Protoc Neurosci. doi:10.1002/0471142301.ns0417s57 and Guo P, et al. (2012) Rapid and simplified purification of recombinant adeno-associated virus. J Virol Methods 183(2):139-146).

Once the virus has been produced in the, e.g., 293 cells, the cells are collected, lysed, and the resultant virus is purified. Density gradient ultracentrifugation, e.g., in cesium chloride or nonionic iodixanol (VISIPAQ™) gradients and column chromatography, such as ion-exchange, heparin-affinity, or mucin-affinity column chromatography, depending on the AAV serotype. Once the rAAV has been purified and concentrated to a suitable concentration, the virus can be used for in vitro cell transduction or for in vivo animal injection at an appropriate MOI (Multiplicity of Infection).

Numerous rAAV vectors have been made containing genes for expressing RNAs and proteins, and are commercially available. A “gene” is a genetic element for production of a gene product such as a protein or RNA. A gene for production of a protein product includes, from to 3′ according to convention: one or more regulatory elements (transcription control elements) such as promoters, transcription response elements (TREs), repressors, enhancers; an open-reading frame (ORF) encoding a protein or a sequence encoding a functional RNA; and a polyadenylation (pA) site. Due to size limitations, genes for use in rAAV vectors typically do not include introns. rAAV vectors also include the 5′ ITR and 3′ ITR flanking the gene, which is referred to as a transgene. Thus a typical rAAV genome has the following structure, in order from 5′ to 3′ on the sense strand: ITR-promoter-transgene-pA-ITR, and in one aspect of the present invention, the promoter includes a TRE and the sequence encoding a functional RNA is that of a miRNA or shRNA. Methods of molecular cloning of rAAV transgene constructs, preparation of rAAV particles, and storage and use thereof are broadly-known and further technical details are unnecessary for one of ordinary skill in the art to be able to construct useful rAAV vectors, and produce and use rAAV particles as described herein. As indicated above, so long as the gene sequence is less than the packaging limit of rAAV or scAAV, it is useful for production of a transduction particle as described herein.

AAV is but one of many robust and well-characterized viral vectors suited for delivery of the MIR29A reagent, which also includes, without limitation, gammaretroviruses, lentiviruses, adenovirus, and herpes simplex virus. While AAV may be preferred in many instances, other safe and effective viral transducing particles can be developed based on the inducible colorimetric genes described herein for use in the devices, systems and methods described herein. Likewise, plasmid or naked DNA, optionally combined with transfection reagents may also be employed. Nevertheless, the high efficiency transduction of safe, recombinant viral particles, such as rAAV particles, are preferred in many instances.

### EXAMPLES

Ligamentum flavum hypertrophy results from multiple complex pathways which are potentially manipulable. The thickness of the ligamentum flavum in stenotic lumbar spines is an age-dependent gender-independent phenomenon. Further, ligamentum flavum hypertrophy is due to the accumulation of inflammation-related scar tissue. Specific substances are implicated in the pathogenic mechanism collagen I, collagen III, and regulators thereof. Interest in the regulation of gene expression programming and phenotypic development has led to the study of microRNAs. MicroRNAs are small noncoding cellular RNA molecules that prevent protein translation by binding to and inhibiting or destroying their messenger RNA targets. MicroRNAs have different effects in different body tissues, serving as a fundamental regulator of expression. One specific type of microRNA, microRNA-29a (miR29), regulates fibrosis in certain tissues. miR29 reportedly decreases the expression of TIMP1 and TGF-β. Notably, reduced plasma levels of miR29a have been directly correlated with ligamentum flavum hypertrophy in patients with spinal stenosis. This raises the question of whether miR-29a a key regulator of ligament flavum hypertrophy.

Lumbar spinal stenosis remains the most common cause for spinal surgery in patients over the age of 65. Currently, non-operative management consists of physical therapy, NSAIDs, and steroid injections. While these strategies target the symptoms of spinal stenosis, they do not directly target the underlying pathology. Based on the data below, microRNA-29a may prove to be a conservative treatment option that may prolong or prevent surgical intervention.

### Example 1

Six patients (3 M, 3 F; age 68±10.8 years) underwent L3-L5 laminectomy to address symptomatic spinal stenosis. LF thickness was measured on preoperative axial T1 MRIs to identify hypertrophic and non-hypertrophic levels. In each patient, hypertrophic LF was collected from L4/L5, which served as an experimental group, and non-hypertrophic LF was removed from L2/L3, which served as a control group. RT-PCR and the comparative ΔΔCt method were performed to establish relative gene expression profiles of collagen 1, collagen 3, and microRNA-29a levels. The measurements of LF thickness, collagen 1, collagen 3, and microRNA-29a levels were compared using paired t-tests. The correlations among collagen I, collagen III, and microRNA-29a levels were analyzed using Pearson's correlation coefficients. p<0.05 was considered statistically significant.

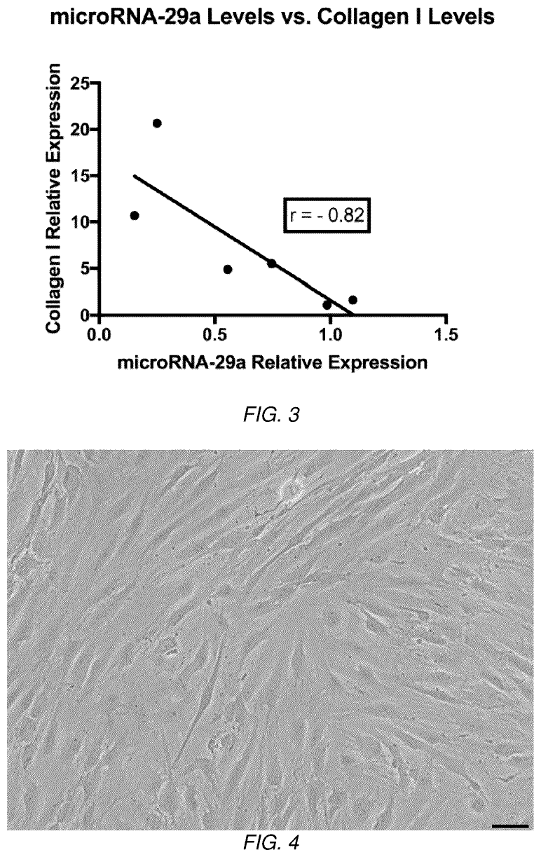

The thickness of LF in the stenotic levels was significantly higher than in the non-stenotic levels (6.8±0.9 mm vs 4.0±0.9 mm, p<0.01). mRNA levels of collagen 1 were significantly higher (p=0.04) and microRNA-29a levels were significantly lower (p=0.03) in hypertrophic LF compared to control LF (FIGS. 2 and 3). The mRNA levels of collagen 3 were higher in the hypertrophic LF, although this was not significant (p=0.10). MicroRNA-29a level was negatively correlated with type I collagen level (r=−0.82, p=0.02) and type III collagen level (r=−0.53, p=0.14).

These data suggest that there is an association between decreased microRNA-29a levels and LF hypertrophy. Follow-on studies may evaluate whether microRNA-29a is protective against LF hypertrophy and whether it may serve as a therapeutic target in the management of LSS. These data suggest that microRNA-29a may potentially serve as a therapeutic strategy for spinal stenosis and be protective against LF hypertrophy and lay the groundwork for further investigations using LF cell cultures to determine whether upregulation of microRNA-29a decreases fibroblast collagen production and whether this acts via repression of TAB1-mediated TIMP-1 production.

### Example 2

The following studies are planned as follow-on work for Example 1.

Study the correlative relationship between tissue levels of microRNA-29a and ligamentum flavum hypertrophy. Ligamentum flavum samples obtained from patients who underwent elective lumbar laminectomy with T1-weighted MRI evidence of ligamentum flavum hypertrophy had ligamentum flavum tissue sampled from hypertrophic and non-hypertrophic levels. Ligamentum flavum thickness was determined using MRI measurement. The ligamentum flavum tissue is examined histologically for collagen and elastin staining. Measurements of microRNA-29a, Collagen I mRNA, and Collagen III mRNA in the sample is determined using quantitative RT-PCR. It is expected that microRNA-29a levels will be lower in ligamentum flavum tissue sampled from hypertrophic levels as compared to ligamentum flavum tissue sampled from non-hypertrophic levels of the same patient.

Determine whether microRNA-29a regulates pro-fibrotic tissue protein translation. Cellular culture of ligamentum flavum samples is performed. Each lineage is divided into three groups: a control, a microRNA-29a silenced (decreased microRNA-29a expression), and a microRNA29—a precursor (increased microRNA-29a expression) group. Cells in the control group contain scrambled miRNA control. The silenced group is transfected via electroporation with a chemically synthetic complementary strand to bind free microRNA-29a. The precursor group is similarly transfected via electroporation with a lentiviral plasmid containing the microRNA-29a sequence. Cellular expression of collagen I and collagen III messenger RNA is assessed using quantitative RT-PCR. It is expected that: 1) microRNA-29a silencer (serving to decrease microRNA29-a expression) plasmid transfection will result in increased collagen I and collagen III messenger RNA expression in a ligamentum flavum tissue cellular culture samples and 2) microRNA-29a precursor (serving to increase microRNA29-a expression) plasmid transfection via electroporation will result in decreased collagen I and collagen III messenger RNA expression in a ligamentum flavum tissue cellular culture samples.

### Materials and Methods:

Subjects: Subjects include 40 adult patients who underwent lumbar (L3-L5) laminectomy to address symptomatic lumbar spinal stenosis with ligamentum flavum hypertrophy unresponsive to conservative measures for at least one month. Ligamentum flavum thickness was measured on pre-operative axial T1 MRIs to identify hypertrophic and non-hypertrophic levels. In each patient, hypertrophic ligamentum flavum was collected from L4/L5, which serves as an experimental group, and non-hypertrophic ligamentum flavum was removed from L2/L3, which serves as a control group. Intra-operative samples from hypertrophic and non-hypertrophic levels were transported from the operating room to the laboratory in separate containers. Epidural fat and residual bone attached to the ligamentum flavum sample were carefully removed with sterile surgical instruments. Ligamentum flavum tissue samples from hypertrophic and non-hypertrophic levels were stored in separate containers. Samples were flash-frozen in liquid nitrogen then stored in our biorepository at −80° C. The frozen ligamentum flavum samples are readily available to thaw and analyze with RT-PCR for microRNA-29a, collagen I, and collagen III gene expression. In addition, increased patient enrollment with new tissue samples is readily available in case additional samples are needed to repeat experiments due to technical error and or tissue degradation/destruction. Additional ligamentum flavum samples may be required for cellular culture, proliferation, and modulation of tissue microRNA-29a.

Inclusion criteria: Patients diagnosed with lumbar spinal stenosis, having failed conservative treatments (supervised physical therapy, anti-inflammatories, and/or corticosteroid injections) who are scheduled to undergo primary lumbar laminectomy with or without fusion are included. Patients are >18 and <80 years of age, with a BMI of 40 or less.

Exclusion criteria: Participants with a history of prior lumbar spine surgery or trauma are excluded. Participants with ossification of the ligamentum flavum defined on MRI as low signal intensity thickening of the ligament in both T1 and T2 sequences on the posterior margin of the spinal canal, causing indentation of the theca with or without cord compression also are excluded.

Ligamentum Flavum Thickness Imaging Measurement: Pre-operative MRIs from all patients included were assessed for evidence of lumbar spinal stenosis and ligamentum flavum hypertrophy. Hypertrophic and non-hypertrophic levels were confirmed. Maximal thickness was measured using a manual cursor method in the picture archiving and communication system (PACS), which was then automatically calculated using the integrated software. Two board-certified radiologists independently performed and reviewed measurements during two different sessions. The average of the two measured values are used. This process is repeated as needed for all newly enrolled subjects.

Ligamentum Flavum Histologic Analysis: Ligamentum flavum samples are examined histologically for collagen and elastin staining. The histology section of the lab houses two cutting workstations, a cryostat, tissue preparation stations, and a paraffin embedder. In addition, the microscopy room includes a Nikon Eclipse TS100 inverted microscope, a Nikon Eclipse E800 fluorescent transmission microscope, a stereo microscope for dissection, and an RTse SPOT camera. Collagen content staining is performed using Masson's trichrome staining.

Ligamentum Flavum Human Tissue Culture Surgical ligamentum flavum specimens will be used for tissue culture. The Ferguson laboratory tissue culture space consists of three sterile hoods, four HERACell 150i incubators, and one ThermoForma Series II Water Jacketed CO₂ incubator. We have previously established a successful protocol for the culture of ligamentum flavum cells. Ligamentum flavum tissue sample is digested in 0.2% pronase (EMD Chemicals 53702) for 60 minutes, followed by overnight digestion in 0.02% collagenase P (Roche Applied Science 11213872001) to obtain isolated ligamentum flavum cells. These primary ligamentum flavum cells are cultured under in hypoxic conditions (37° C., 5% CO₂, 5% O₂, and in buffered F12 medium supplemented with 10% FBS) (FIG. 4).

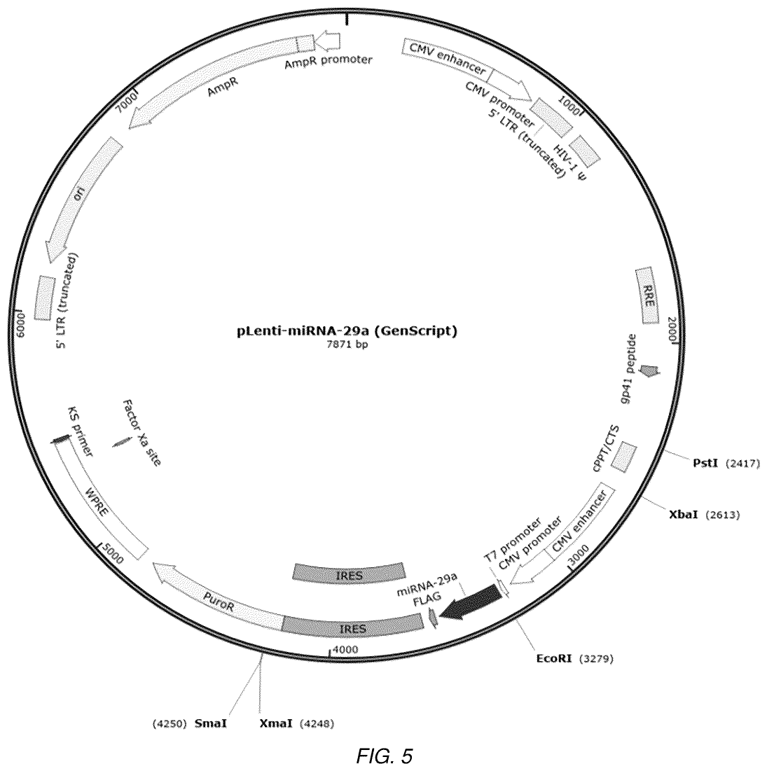

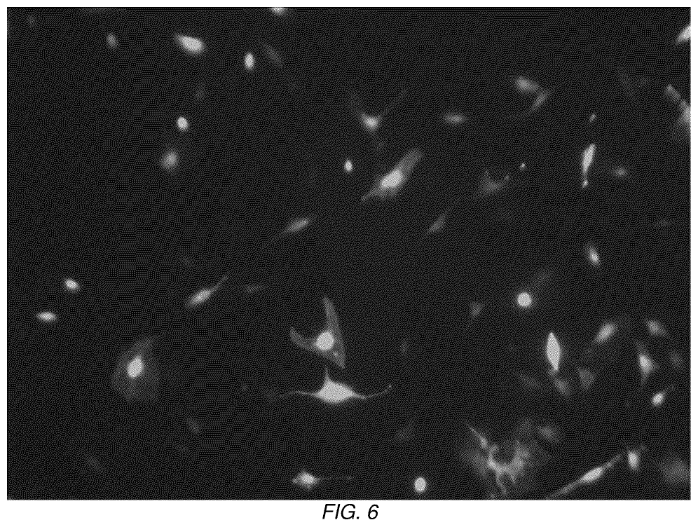

Oligonucleotides, Constructs, and Transfections: MicroRNA-29a lentiviral plasmid precursor (FIG. 5) was purchased from GenScript Biotech (Piscataway, NJ) to overexpress microRNA-29a and the MISSION® (St. Louis, MO) synthetic microRNA inhibitor, and human hsa-miR-29a-3p was purchased to decrease the level of microRNA-29a. Transfection will be performed using an Amaxa™ Nucleofector™ 2b electroporation system available in the Ferguson laboratory. Our lab has established an electroporation protocol for ligamentum flavum cells modified for maximal cell viability and transfection success. The protocol has approximately 80% viability and 70% transfection efficiency (FIG. 6).

Reverse Transcription (RT) and Quantitative Real-Time PCR (qPCR): PCR is utilized to quantify relative gene expression of microRNA-29a, collagen I, and collagen III for use in quantifying pre-fibrotic genes and microRNA-29a expression relative to housekeeper gene (GADPH) expression. Genes of interest to be explored in ligamentum flavum samples from subjects who underwent lumber laminectomy for spinal stenosis. Collagen primers have been purchased from Invitrogen Thermo Fisher Scientific (Waltham, MA). PCR is performed using a Bio-Rad iQ5 PCR system.

Statistical Analysis: For collagen I, collagen III, and microRNA-29a level relative gene expression profile analysis, the comparative ΔΔCt method is used for quantitative real-time RT-PCR23. This method utilizes the number of cycle thresholds (Ct) which is the value at which signal generated by PCR product can be detected from background noise. The ΔΔCt is the relative change in Ct from the experimental condition compared to the control condition. Quantitative gene expression data is normalized to the expression of housekeeping gene glyceraldehyde-3-phosphate dehydrogenase (GAPDH). For the ligamentum flavum thickness, measurements performed by two independent orthopaedic spine physicians during two different sessions is assessed for absolute values of inter-observer reliability and intra-observer reliability. A Cohen's kappa statistic is calculated for intra-observer and interobserver reliability. An acceptable value is a Cohen's kappa statistic of >0.4. The measurements of ligamentum flavum thickness, collagen I gene expression, collagen III gene expression, and microRNA-29a levels at hypertrophic and non-hypertrophic levels is compared using paired t-test for these continuous variables in the same subjects. To assess the potential linear relationship between microRNA-29a levels, collagen I gene expression, and collagen III gene expression, Pearson's correlation coefficients are calculated. A p-value <0.05 is considered statistically significant for all data with relevant statistical significance testing.

Estimation of Sample Size and Power: This study's estimated sample size and power for detecting a correlative relationship between tissue levels of microRNA-29a and ligamentum flavum hypertrophy and microRNA-29a modulation of gene expression was calculated using the online UCSF sample size calculator for clinical trials and Power Calculations Program (PS Version 3.1.2). This estimation was calculated using pilot study data. If the true correlation of collagen III relative gene expression and microRNA-29a relative gene expression has a correlation coefficient of r=|0.53| with an alpha of 0.05 and beta of 0.20 then a total sample size of 26 would be necessary. A study of a continuous response variable from matched pairs of study subjects is planned. Prior data indicate that the difference in the response of matched pairs is normally distributed with a standard deviation of 28.13. If the true difference in the mean response of matched pairs is 44.77, five pairs of subjects will need to be studied to be able to reject the null hypothesis that this response difference is zero with probability (power) 0.8. The Type I error probability associated with this test of this null hypothesis is 0.05. Both studies are adequately powered with a sample size of 40 subjects.

Expected Findings and Possible Interpretations: It is predicted that microRNA-29a transfected cells that over-express microRNA-29a will decrease relative gene expression of collagen I and collagen III as compared to controls. MicroRNA-29a silencer transfected cells that under-express microRNA-29a are expected to have increased relative gene expression of collagen I and III as compared to controls. These findings would indicate microRNA-29a as a potential therapeutic target in the management of ligamentum hypertrophy-related spinal stenosis.

### Example 3

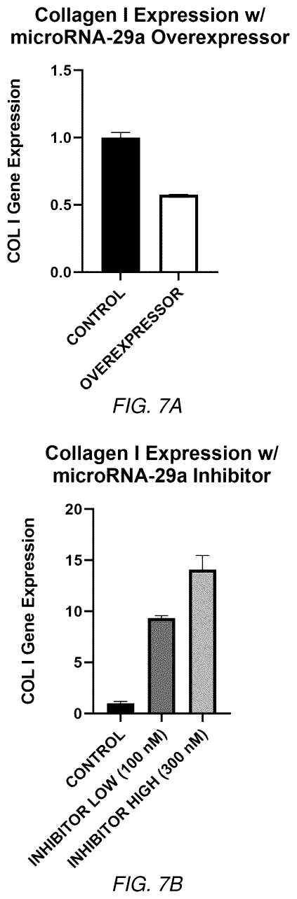

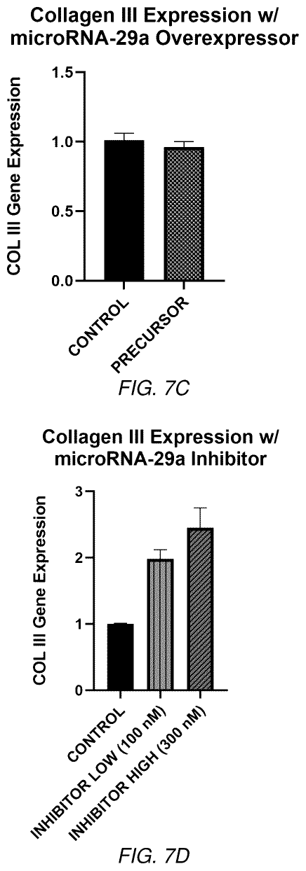

Non-hypertrophic LF cells were transfected in vitro, essentially as described in Example 2 with the Lentiviral construct depicted in FIG. 3 to produce overexpressor cells. Alternatively, non-hypertrophic LF cells were transfected in vitro Amaxa® Human Chondrocyte Nucleofector® Kit (Lonza) with an antisense microRNA-29a inhibitor (a 2′-O-methylated RNA duplex version of hsa-miR-29a-3p MIMAT0000086: 5′-uagcaccaucugaaaucgguua-3′ (SEQ ID NO: 4), MISSION® microRNA Mimic, has-miR-126, HMI0117, Sigma-Aldrich). RT-PCR was conducted for collagen I & III expression after in-vitro transfection with the microRNA-29a inhibitor or overexpressor. The MicroRNA-29a inhibitor increased collagen I & III expression. MicroRNA-29a inhibition using a synthetic double-stranded antisense RNA competitive inhibitor at 100 nanomolar dosing resulted in nearly a nearly 10-fold increase in collagen I expression relative to control (FIG. 7B). The 300 nanomolar dose resulted in a nearly 15-fold increase in the relative gene expression of collagen I (FIG. 7B). Similarly, for collagen III the same competitive inhibitor at 100 nanomolar dosing resulted in double the collagen III expression relative to control (FIG. 7D). The 300 nanomolar dose resulted in 2.5 times the relative gene expression of collagen III (FIG. 7D). Over-expression of MicroRNA-29a using a lentiviral plasmid containing precursor sequences for microRNA-29a resulted in nearly half the relative gene expression of collagen I (FIG. 7A). Over-expression of MicroRNA-29a did not appreciably alter collagen III gene expression in these preliminary experiments (FIG. 7C). In conclusion, we found sufficient evidence to support the hypothesis that MicroRNA-29a suppresses ligamentum flavum hypertrophy through inhibition of collagen I expression. Evidence pertaining to collagen III is inconclusive. The above supports the conclusion that microRNA-29a is believed to be useful in the management of lumbar spinal stenosis.

### Example 4: 3D Culture System to Model the Ligamentum Flavum in Health and Disease

Lower back pain (LBP) is the leading cause of years-lost-to-disability, costing an estimated 0.1-2% of GDP in industrialized countries. Lumbar spinal stenosis (LSS) is a prevalent and disabling cause of LBP, affecting an estimated 30 million persons in the United States alone.

Laminectomies are among the most common procedures for LSS, indicating the prevalence of ligamentum flavum hypertrophy (LFH) in LSS. Although cases of LSS are successfully managed palliatively, 4-600,000 patients per year develop debilitating chronic disease requiring surgery, suffering high morbidity and incurring high costs to the medical system. Reducing LFH and the pain and disability associated with it will greatly improve quality of life of these patients and the burden on LBP on healthcare systems. The etiology and pathogenesis of the disease is poorly understood. Studies of human diseased tissue and animal models have shown that LFH is characterized by vascular and immune cell invasion concomitant with dramatic changes in the tissue extracellular matrix. Specifically, one observes: a decrease in elastin and increases in (COL1) and collagen III (COL3) production. The regulation of gene expression and phenotypic development of LF fibrosis is the result of complex cell and tissue interactions under the influence of many regulatory pathways. Recent molecular analyses have shown that miRNA-29a (miR29a) is a crucial regulator of fibrosis. miR29a reportedly decreases the expression of TIMP1 and TGF-β, proteins associated with fibrosis and LFH progression and reduced plasma levels of miR29a have been directly correlated with LFH in patients with spinal steno sis. Preliminary data demonstrate that over-expression of miR29a in LF cells grown in monolayer can significantly reduce COL1 gene expression. The goal of the following is to develop a 3D model of the Ligamentum Flavum to test the ability of miR29a to slow or reverse phenotypic changes observed in LFH. To this end, a 3D culture model for LF studies is provided. It is believed that LF cells in 3D culture will produce more extracellular matrix (ECM) than cells in 2D monolayer on tissue-culture plastic. It is expected that higher expression of COL1, COL3, and Elastin, along with an increase in Col1/Col3 and Col1/Elastin ratios, will be observed in 3D cultures. Further, exposure to the inflammatory factor IL1-1b will decrease the expression of ECM genes.

### Methods

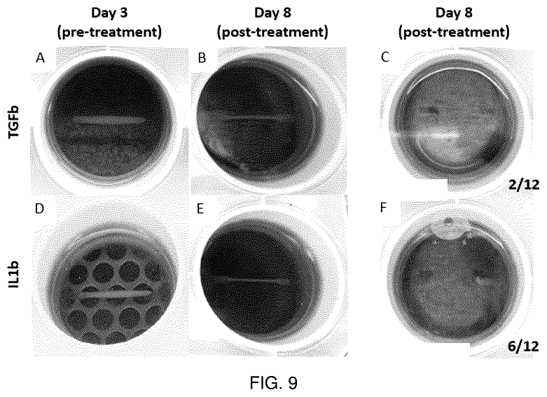

Primary human cells from the ligamentum flavum obtained as surgical waste from patients undergoing posterior decompression surgery. Tissue was minced to 1-2 mm portions, digested for 16-18 hours in collagenase type 1 (Worthington), and cultured and expanded in basal medium (DMEM-high glucose, 10% fetal bovine serum (FBS), 100 units/ml penicillin and 100 mg/ml streptomycin, Gibco). All cells were utilized at passage 1-2. Referring to FIG. 9, 3D cultures under static tension were formed in a FlexCell™ Tissue-Train™ plates. LF cells were encapsulated within 2% collagen (EZ-COL™, BioMatrix) using the Flexcell Tissue Train Culture System (Flexcell, Inc,) and re-differentiated for 7 days in serum-free tissue-specific medium containing 10 ng/ml TGFb1. After 7 days of re-differentiation, the LF models were exposed to one of 3 different conditions: 10 ng/ml TGFb1, 10 ng/ml IL-1b or both TGFb1 and IL-1b for 24 hours. In further detail, LF model cultured in Tissue-Train™ plates with TGFb or IL1b for three days or 8 days that includes exposure to TGFb or IL1b treatment for the final 24 hours. The 3D LF Model macroscopic appearance of each culture well was evaluated. At day 8, most samples treated were still contiguous. After 24 hours of TGFb treatment, 2/12 samples had ruptured as compared to 6/12 rupturing with 24 hours of IL1b treatment. Samples were processed for RT-PCR analysis of the expression of COL1, COL3, and Elastin.

### Results

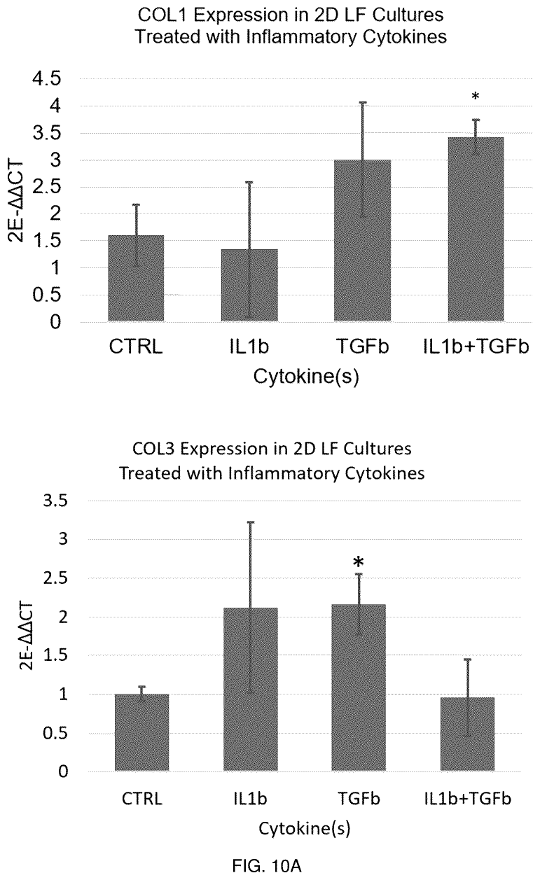

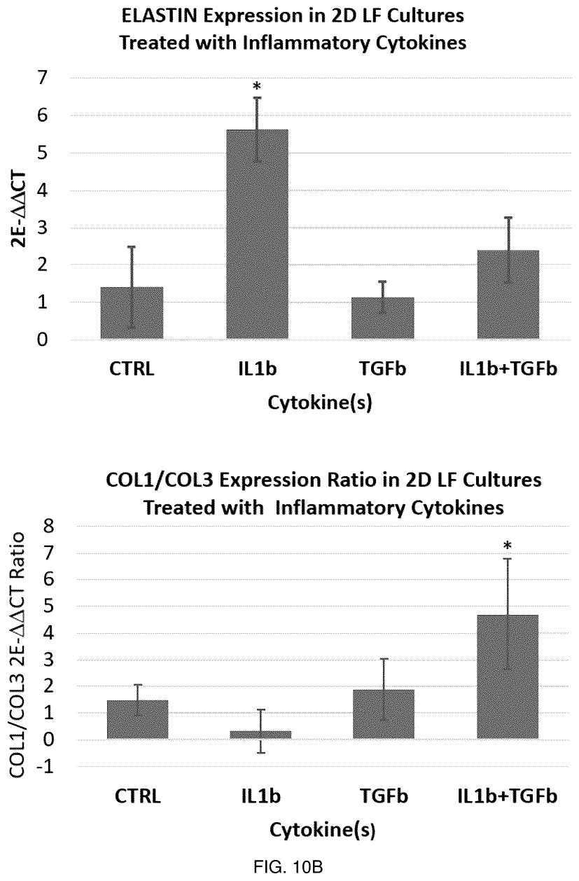

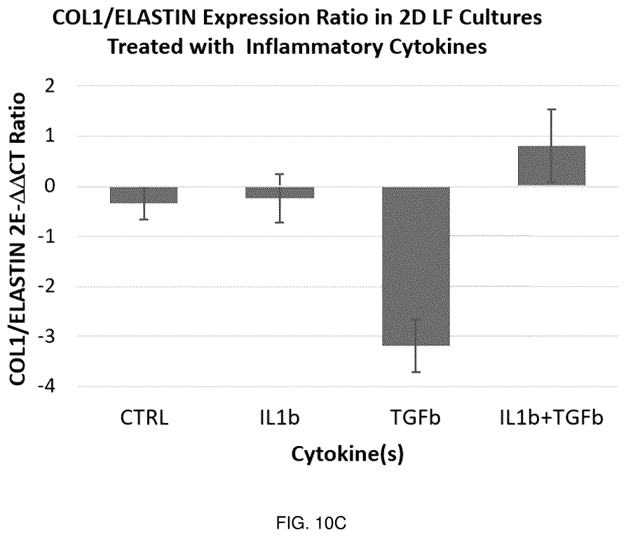

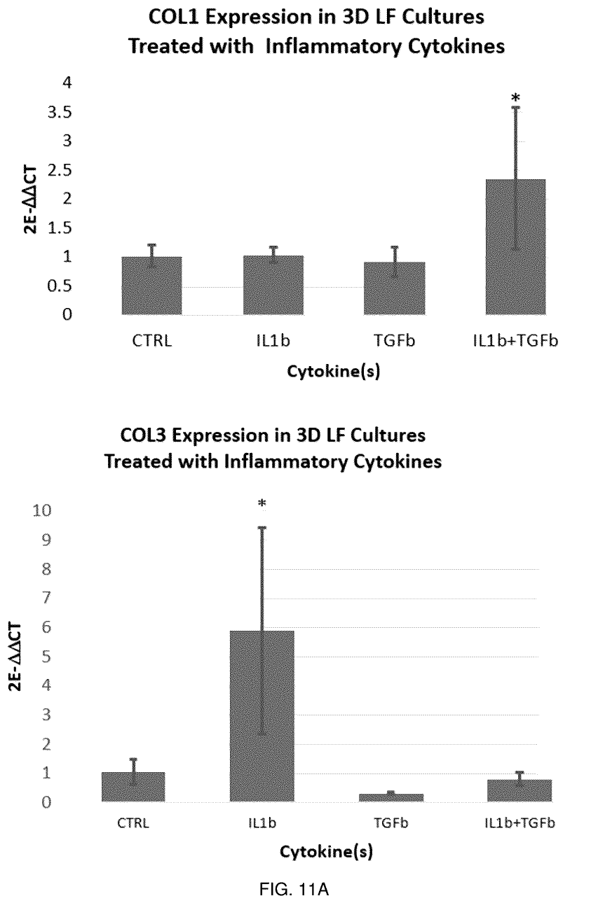

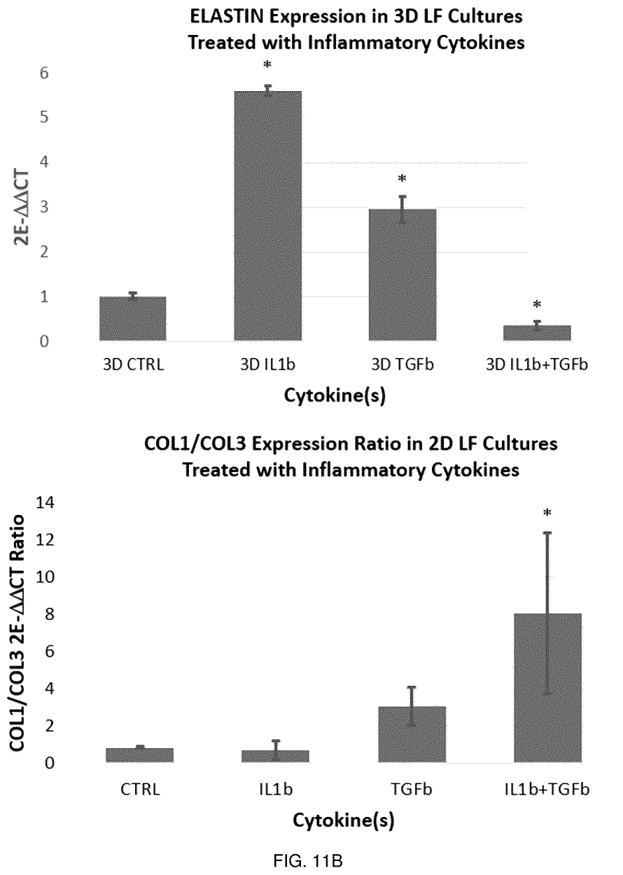

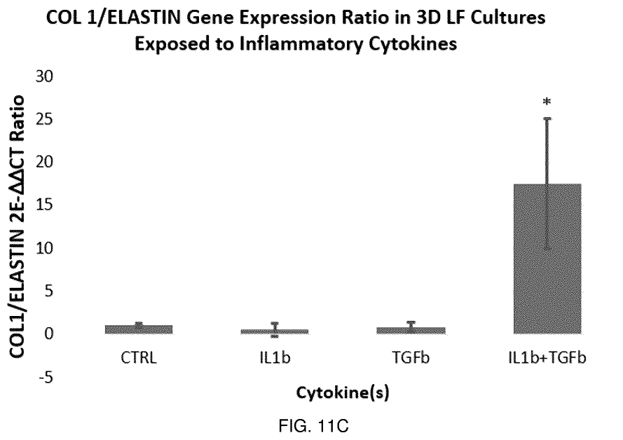

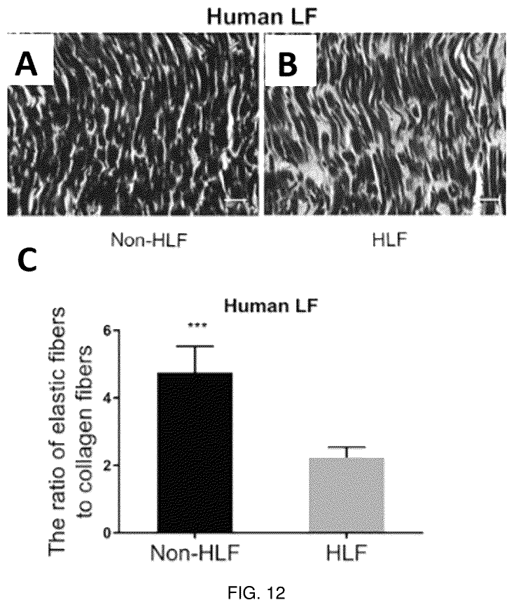

Human primary LF cells encapsulated within Collagen 1 (rat tail) hydrogels respond to inflammatory cytokines (See, FIGS. 10A-10C and 11A-11C). In monolayer, TGFb1 induced the expected increase in COL1 and COL3. However, the COL1/COL3 gene expression ratio did not with TGFb1 (hypertrophy). In monolayer, IL1b had a different effect on the LF cells, inducing an increase in elastin expression, but not COL 1 or COL3. The combination of TGFb1 and IL1b induced a robust increase in the COL1/COL3 ratio. In 3D culture, the patterns of COL1, COL3 and Elastin gene expression with TGFb1 and IL1b were maintained. In 3D culture, the combination of TGFb1 and IL1b induced an increase in the COL1/Elastin ratio as well, where reduction in Elastin is a hallmark of the disease in the clinic (FIG. 12). As such, a unique model of LH hypertrophy using primary human LF cells in a hydrogel under tension is provided. With this model, the efficacy of novel therapeutics to reduce LFH can be tested.

Efficacy of a MIR29A reagent according to any embodiment described herein may be ascertained by delivering the MIR29A reagent in varying amounts to the described primary human LF cells of culture system, for example by transfection, e.g., by contacting the cells with a MIR29A reagent associated with a lipid nanoparticle, or any other effective delivery method. Optimal dosage windows for MIR29A reagents can be determined using this culture system.

### Example 5—Rat LFH Model

Current models of LFH are suboptimal with considerable barriers. Existing mechanical overload and non-invasive bipedal models require unique equipment and facilities and extensive time, husbandry and resource investment that still result in variable outcomes, while reported surgical models are faster, less expensive and more consistent in outcome but involve the technically difficult and highly traumatic abrasion of lumbar spinal facets joints and transection of numerous ligaments and muscles that render the interpretation of results difficult.

The rat research model has a long history of use in behavioral studies, key to assessing pain, and has other advantages including its well-characterized physiology, a growing list of genetic tools for mechanistic analysis, a size amenable to surgical manipulation and low husbandry costs. The goal is to make important refinements to the reported rat instability model to increase reproducibility and use it to examine LFH pathogenesis overtime using single cell RNA sequencing (scRNAseq) and validate the rat model by comparing it to new scRNAseq data from healthy and hypertrophic human LF samples.

A rat model of LHF was recently reported (see, Wang B, et al. The increased motion of lumbar induces ligamentum flavum hypertrophy in a rat model. BMC Musculoskelet Disord. 2021 Apr. 6; 22(1):334). Three improvements including a useful accelerant are proposed. First, the superior and inferior articular (facet) processes are resected for a clean wound to prevent fusion instead of partially grinding the articular surfaces that can create a regenerative environment, resulting result in fusion. Second, the intersegmental musculature removed in the recently reported study is preserved in favor of a third modification: removal of the pars articularis, which together will increase instability of the vertebral segment further without unnecessary trauma. In the characterization of LFH pathogenesis, analysis of LFH in humans and animals has been restricted to, at its most granular, bulk genome-wide association analysis (GWAS) at the disease endpoint. While there is a clear change in gene expression and cell content, i.e. the emergence and proliferation of vascular cell types, macrophages and myofibroblasts, very little is known of the origin of myofibroblast, the cell interactions that drive scar formation and the altered LF cell phenotype, prohibiting the development of therapies for the prevention and early treatment of LFH. scRNAseq has been initiated for characterizing IDD, and the tools and expertise exist to perform the same analysis in LFH pathogenesis.

Animals Brown Norway Fisher 344 rats from both genders may be obtained from Charles River at 6 months of age. All rats may be housed in a temperature- and humidity-controlled environment with a 12 h light/12 h dark cycle. Prior to surgery, animals may be randomly divided into one of three groups: Group 1, the described, refined surgically-induced spine instability LFH model; Group 2, the original, reported spine instability LFH model (Wang, et al.); and Group 3, sham surgery only.

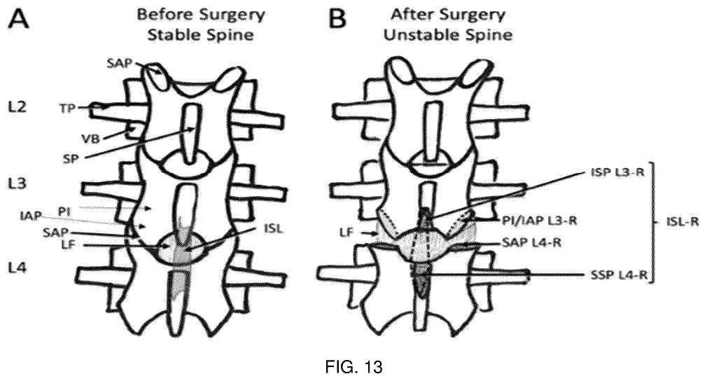

Surgery Animals may be anesthetized with 3% isoflurane and 02 gas (1.5 L/min) delivered through an inhalation mask. The surgical area is aseptically prepared by depilatory hair removal, betadine and subsequent 70% ethanol scrub repeated 3×. A midline incision is made between L1 and S1. Muscles are separated to expose the posterior vertebral elements and the ligamentum flavum. In Groups 1-3, soft tissue and capsule are removed from the facet joints. In Group 1, bone cutting forceps are used to cleanly and bluntly remove the anterior and posterior elements of the facet joint and the spinous processes (and interspinous ligament) between L3 and L4. Then the pars articularis is exposed by flexion of the spine and cut away using bone cutting forceps. The paraspinal muscles are maintained. In Group 2, a burr may be used to partially grind the facet joint surfaces, the interspinous processes removed as in Group 1 and the intersegmental musculature removed. In Groups 3, only surgical exposure occurs. In all groups, the wounds are closed in routine fashion. A single dose of ketoprofen may be given to prevent pain from the injection. It is anticipated that ketoprofen may be used for no more than 3 days after injection. The animal may be placed a holding chamber until it ambulates normally when it is then returned to its box. See, FIG. 13 for additional details.

Husbandry All animals may be kept in standard cage conditions with bedding cages with free access to food and water.

Analysis All 6 animals from Groups 1-3 are harvested after 8 weeks of surgery and free movement. The affected segment (L3/L4) is separated from the healthy (unaffected) segments (L2/L3 and L4/L5) by transection of the 3^{rd} and 4^{th} vertebral bodies. The entire vertebral segments (L2/L3, L3/L4, and L5/L5) are fixed in 4% paraformaldehyde for 24 hours at 4° C. before they undergo 72 hours of decalcification in ImmunoCal (Immunotec). After decalcification, the samples are processed for paraffin embedding. All samples may be serial sectioned sagittally and selected samples processed with histochemical or immunohistochemical staining. Histochemical staining histological and morphological characteristic of the LF may be assessed by H&E, Mallory's Trichrome and Verhoeff's (to ID elastin and collagens) and Pico-sirius red (for collagen fibril alignment); and (2) immunohistochemistry for signs of LFH including COL1, COL3 and TGFβ1, HIC1, macrophage state (naïve, M1 and M2) and myofibroblast invasion by α-SMA.

Efficacy of MIR29A reagent according to any embodiment described herein may be ascertained by delivering the MIR29A reagent to ligamentum flavum cells of the animals as described herein, for example by transfection, e.g., by contacting the cells with a MIR29A reagent associated with a lipid nanoparticle. Optimal dosage windows for MIR29A reagents can be determined using this culture system.

The present invention has been described with reference to certain exemplary embodiments, dispersible compositions and uses thereof. However, it will be recognized by those of ordinary skill in the art that various substitutions, modifications or combinations of any of the exemplary embodiments may be made without departing from the spirit and scope of the invention. Thus, the invention is not limited by the description of the exemplary embodiments.

1. A method of treating spinal stenosis in a patient, treating hypertrophic LF in a patient, and/or reducing expression of type I and/or III collagen, and/or treating fibrosis in LF of a patient, comprising delivering to cells of a patient's ligamentum flavum (LF) an amount of a MIR29A reagent effective to treat spinal stenosis in the patient, to treat hypertrophic LF in the patient, or to reduce expression of type I and/or III collagen, and/or treat fibrosis in LF in the patient.

2. The method of claim 1, wherein the patient has hypertrophic LF, and the MIR29A reagent is delivered to cells of the hypertrophic LF.

3. The method of claim 1, wherein the MIR29A reagent is delivered by transforming a cell of the LF of the patient with a nucleic acid comprising a gene for expressing the MIR29A reagent.

4. The method of claim 3, wherein the nucleic acid is a recombinant viral genome, optionally delivered to the cell in a viral particle or in a liposome particle.

5. The method of claim 4, wherein the recombinant viral genome is an Adeno-associated Virus (AAV) genome, optionally packaged in a viral transducing unit.

6. The method of claim 1, wherein the MIR29A reagent is conspecific to the patient.

7. The method of claim 1, wherein the MIR29A reagent is delivered in a pharmaceutical composition by a nanocarrier, that optionally is selected from a liposome, a lipid nanoparticle, an exosome, a dendrimer, or a polymer particle.

8. The method of claim 1, wherein the LF is injected directly with the MIR29A reagent by epidural delivery.

9. The method of claim 1, wherein the MIR29A reagent is a microRNA-29a precursor RNA, optionally having the sequence: augacugauuucuuuugguguucagagucaauauaauuuucuagcaccaucugaaaucgguuau (SEQ ID NO: 2), or a mature microRNA-29a RNA, optionally comprising either one or both of:
hsa-miR-29a-5p MIMAT0004503: 5′-acugauuucuuuugguguucag-3′ (SEQ ID NO: 3); and
hsa-miR-29a-3p MIMAT0000086: 5′-uagcaccaucugaaaucgguua-3′ (SEQ ID NO: 4).

10. The method of claim 1, wherein the patient is a human patient.

11. A method of treating spinal stenosis in a patient or of reducing expression of type I collagen, reducing expression or type III collagen, and/or treating fibrosis in LF of a patient, comprising delivering to cells of a patient's ligamentum flavum (LF) an amount of a reagent for knocking down type I collagen expression in the cells effective to treat spinal stenosis in the patient or to reduce expression of type I collagen in the cells and/or treat fibrosis in LF in the patient and/or an amount of a reagent for knocking down type III collagen expression in the cells effective to treat spinal stenosis in the patient or to reduce expression of type III collagen in the cells and/or treat fibrosis in LF in the patient.

12. The method of claim 11, wherein the patient has hypertrophic LF, and the reagent is delivered to cells of the hypertrophic LF.

13. A method of treating hypertrophic LF in a patient, comprising delivering to cells of a patient's ligamentum flavum (LF), e.g., transfecting cells of the patient's LF with, an amount of a reagent for knocking down type I collagen expression in the cells effective to treat hypertrophic LF in the patient and/or an amount of a reagent for knocking down type III collagen expression in the cells effective to treat hypertrophic LF in the patient.

14. The method of claim 13, wherein the reagent is delivered to cells of the hypertrophic LF.

15. The method of claim 13, wherein the reagent is conspecific to the patient.

16. The method of claim 13, wherein the reagent is delivered in a pharmaceutical composition by a nanocarrier, such as a liposome, a lipid nanoparticle, an exosome, a dendrimer, or a polymer particle.

17. The method of claim 13, wherein the LF is injected directly with the reagent by epidural delivery.

18. The method of claim 13, wherein the reagent is an antisense reagent or an RNAi reagent.

19. The method of claim 13, wherein the patient is a human patient.


SEQUENCE LISTING (7 sequences)

SEQ ID NO: 1 - DNA, 64 bp, Homo sapiens
  source 1..64: mol_type = other DNA
atgactgatttcttttggtgttcagagtcaatataattttctagcaccatctgaaatcggttat

SEQ ID NO: 2 - RNA, 64 nt, Homo sapiens
  source 1..64: mol_type = other RNA
atgactgatttcttttggtgttcagagtcaatataattttctagcaccatctgaaatcggttat

SEQ ID NO: 3 - RNA, 22 nt, Homo sapiens
  source 1..22: mol_type = other RNA
actgatttcttttggtgttcag

SEQ ID NO: 4 - RNA, 22 nt, Homo sapiens
  source 1..22: mol_type = other RNA
tagcaccatctgaaatcggtta

SEQ ID NO: 5 - DNA, 20 bp, synthetic construct
  source 1..20: mol_type = other DNA
actgtcttcttggccatgcg

SEQ ID NO: 6 - DNA, 5914 bp, Homo sapiens
  source 1..5914: mol_type = other DNA
gcagacgggagtttctcctcggggtcggagcaggaggcacgcggagtgtgaggccacgcatgagcggacgctaaccccctccccagccacaaagagtctacatgtctagggtctagacatgttcagctttgtggacctccggctcctgctcctcttagcggccaccgccctcctgacgcacggccaagaggaaggccaagtcgagggccaagacgaagacatcccaccaatcacctgcgtacagaacggcctcaggtaccatgaccgagacgtgtggaaacccgagccctgccggatctgcgtctgcgacaacggcaaggtgttgtgcgatgacgtgatctgtgacgagaccaagaactgccccggcgccgaagtccccgagggcgagtgctgtcccgtctgccccgacggctcagagtcacccaccgaccaagaaaccaccggcgtcgagggacccaagggagacactggcccccgaggcccaaggggacccgcaggcccccctggccgagatggcatccctggacagcctggacttcccggaccccccggaccccccggacctcccggaccccctggcctcggaggaaactttgctccccagctgtcttatggctatgatgagaaatcaaccggaggaatttccgtgcctggccccatgggtccctctggtcctcgtggtctccctggcccccctggtgcacctggtccccaaggcttccaaggtccccctggtgagcctggcgagcctggagcttcaggtcccatgggtccccgaggtcccccaggtccccctggaaagaatggagatgatggggaagctggaaaacctggtcgtcctggtgagcgtgggcctcctgggcctcagggtgctcgaggattgcccggaacagctggcctccctggaatgaagggacacagaggtttcagtggtttggatggtgccaagggagatgctggtcctgctggtcctaagggtgagcctggcagccctggtgaaaatggagctcctggtcagatgggcccccgtggcctgcctggtgagagaggtcgccctggagcccctggccctgctggtgctcgtggaaatgatggtgctactggtgctgccgggccccctggtcccaccggccccgctggtcctcctggcttccctggtgctgttggtgctaagggtgaagctggtccccaagggccccgaggctctgaaggtccccagggtgtgcgtggtgagcctggcccccctggccctgctggtgctgctggccctgctggaaaccctggtgctgatggacagcctggtgctaaaggtgccaatggtgctcctggtattgctggtgctcctggcttccctggtgcccgaggcccctctggaccccagggccccggcggccctcctggtcccaagggtaacagcggtgaacctggtgctcctggcagcaaaggagacactggtgctaagggagagcctggccctgttggtgttcaaggaccccctggccctgctggagaggaaggaaagcgaggagctcgaggtgaacccggacccactggcctgcccggaccccctggcgagcgtggtggacctggtagccgtggtttccctggcgcagatggtgttgctggtcccaagggtcccgctggtgaacgtggttctcctggccctgctggccccaaaggatctcctggtgaagctggtcgtcccggtgaagctggtctgcctggtgccaagggtctgactggaagccctggcagccctggtcctgatggcaaaactggcccccctggtcccgccggtcaagatggtcgccccggacccccaggcccacctggtgcccgtggtcaggctggtgtgatgggattccctggacctaaaggtgctgctggagagcccggcaaggctggagagcgaggtgttcccggaccccctggcgctgtcggtcctgctggcaaagatggagaggctggagctcagggaccccctggccctgctggtcccgctggcgagagaggtgaacaaggccctgctggctcccccggattccagggtctccctggtcctgctggtcctccaggtgaagcaggcaaacctggtgaacagggtgttcctggagaccttggcgcccctggcccctctggagcaagaggcgagagaggtttccctggcgagcgtggtgtgcaaggtccccctggtcctgctggtccccgaggggccaacggtgctcccggcaacgatggtgctaagggtgatgctggtgcccctggagctcccggtagccagggcgcccctggccttcagggaatgcctggtgaacgtggtgcagctggtcttccagggcctaagggtgacagaggtgatgctggtcccaaaggtgctgatggctctcctggcaaagatggcgtccgtggtctgactggccccattggtcctcctggccctgctggtgcccctggtgacaagggtgaaagtggtcccagcggccctgctggtcccactggagctcgtggtgcccccggagaccgtggtgagcctggtccccccggccctgctggctttgctggcccccctggtgctgacggccaacctggtgctaaaggcgaacctggtgatgctggtgctaaaggcgatgctggtccccctggccctgccggacccgctggaccccctggccccattggtaatgttggtgctcctggagccaaaggtgctcgcggcagcgctggtccccctggtgctactggtttccctggtgctgctggccgagtcggtcctcctggcccctctggaaatgctggaccccctggccctcctggtcctgctggcaaagaaggcggcaaaggtccccgtggtgagactggccctgctggacgtcctggtgaagttggtccccctggtccccctggccctgctggcgagaaaggatcccctggtgctgatggtcctgctggtgctcctggtactcccgggcctcaaggtattgctggacagcgtggtgtggtcggcctgcctggtcagagaggagagagaggcttccctggtcttcctggcccctctggtgaacctggcaaacaaggtccctctggagcaagtggtgaacgtggtccccctggtcccatgggcccccctggattggctggaccccctggtgaatctggacgtgagggggctcctggtgccgaaggttcccctggacgagacggttctcctggcgccaagggtgaccgtggtgagaccggccccgctggaccccctggtgctcctggtgctcctggtgcccctggccccgttggccctgctggcaagagtggtgatcgtggtgagactggtcctgctggtcccgccggtcctgtcggccctgttggcgcccgtggccccgccggaccccaaggcccccgtggtgacaagggtgagacaggcgaacagggcgacagaggcataaagggtcaccgtggcttctctggcctccagggtccccctggccctcctggctctcctggtgaacaaggtccctctggagcctctggtcctgctggtccccgaggtccccctggctctgctggtgctcctggcaaagatggactcaacggtctccctggccccattgggccccctggtcctcgcggtcgcactggtgatgctggtcctgttggtccccccggccctcctggacctcctggtccccctggtcctcccagcgctggtttcgacttcagcttcctgccccagccacctcaagagaaggctcacgatggtggccgctactaccgggctgatgatgccaatgtggttcgtgaccgtgacctcgaggtggacaccaccctcaagagcctgagccagcagatcgagaacatccggagcccagagggcagccgcaagaaccccgcccgcacctgccgtgacctcaagatgtgccactctgactggaagagtggagagtactggattgaccccaaccaaggctgcaacctggatgccatcaaagtcttctgcaacatggagactggtgagacctgcgtgtaccccactcagcccagtgtggcccagaagaactggtacatcagcaagaaccccaaggacaagaggcatgtctggttcggcgagagcatgaccgatggattccagttcgagtatggcggccagggctccgaccctgccgatgtggccatccagctgaccttcctgcgcctgatgtccaccgaggcctcccagaacatcacctaccactgcaagaacagcgtggcctacatggaccagcagactggcaacctcaagaaggccctgctcctccagggctccaacgagatcgagatccgcgccgagggcaacagccgcttcacctacagcgtcactgtcgatggctgcacgagtcacaccggagcctggggcaagacagtgattgaatacaaaaccaccaagacctcccgcctgcccatcatcgatgtggcccccttggacgttggtgccccagaccaggaattcggcttcgacgttggccctgtctgcttcctgtaaactccctccatcccaacctggctccctcccacccaaccaactttccccccaacccggaaacagacaagcaacccaaactgaaccccctcaaaagccaaaaaatgggagacaatttcacatggactttggaaaatatttttttcctttgcattcatctctcaaacttagtttttatctttgaccaaccgaacatgaccaaaaaccaaaagtgcattcaaccttaccaaaaaaaaaaaaaaaaaaagaataaataaataactttttaaaaaaggaagcttggtccacttgcttgaagacccatgcgggggtaagtccctttctgcccgttgggcttatgaaaccccaatgctgccctttctgctcctttctccacaccccccttggggcctcccctccactccttcccaaatctgtctccccagaagacacaggaaacaatgtattgtctgcccagcaatcaaaggcaatgctcaaacacccaagtggcccccaccctcagcccgctcctgcccgcccagcacccccaggccctgggggacctggggttctcagactgccaaagaagccttgccatctggcgctcccatggctcttgcaacatctccccttcgtttttgagggggtcatgccgggggagccaccagcccctcactgggttcggaggagagtcaggaagggccacgacaaagcagaaacatcggatttggggaacgcgtgtcaatcccttgtgccgcagggctgggcgggagagactgttctgttccttgtgtaactgtgttgctgaaagactacctcgttcttgtcttgatgtgtcaccggggcaactgcctgggggcggggatgggggcagggtggaagcggctccccattttataccaaaggtgctacatctatgtgatgggtggggtggggagggaatcactggtgctatagaaattgagatgcccccccaggccagcaaatgttcctttttgttcaaagtctatttttattccttgatatttttctttttttttttttttttttgtggatggggacttgtgaatttttctaaaggtgctatttaacatgggaggagagcgtgtgcggctccagcccagcccgctgctcactttccaccctctctccacctgcctctggcttctcaggcctctgctctccgacctctctcctctgaaaccctcctccacagctgcagcccatcctcccggctccctcctagtctgtcctgcgtcctctgtccccgggtttcagagacaacttcccaaagcacaaagcagtttttccccctaggggtgggaggaagcaaaagactctgtacctattttgtatgtgtataataatttgagatgtttttaattattttgattgctggaataaagcatgtggaaatgacccaaa

SEQ ID NO: 7 - DNA, 5490 bp, Homo sapiens
  source 1..5490: mol_type = other DNA
ggctgagttttatgacgggcccggtgctgaagggcagggaacaacttgatggtgctactttgaactgcttttcttttctcctttttgcacaaagagtctcatgtctgatatttagacatgatgagctttgtgcaaaaggggagctggctacttctcgctctgcttcatcccactattattttggcacaacaggaagctgttgaaggaggatgttcccatcttggtcagtcctatgcggatagagatgtctggaagccagaaccatgccaaatatgtgtctgtgactcaggatccgttctctgcgatgacataatatgtgacgatcaagaattagactgccccaacccagaaattccatttggagaatgttgtgcagtttgcccacagcctccaactgctcctactcgccctcctaatggtcaaggacctcaaggccccaagggagatccaggccctcctggtattcctgggagaaatggtgaccctggtattccaggacaaccagggtcccctggttctcctggcccccctggaatctgtgaatcatgccctactggtcctcagaactattctccccagtatgattcatatgatgtcaagtctggagtagcagtaggaggactcgcaggctatcctggaccagctggccccccaggccctcccggtccccctggtacatctggtcatcctggttcccctggatctccaggataccaaggaccccctggtgaacctgggcaagctggtccttcaggccctccaggacctcctggtgctataggtccatctggtcctgctggaaaagatggagaatcaggtagacccggacgacctggagagcgaggattgcctggacctccaggtatcaaaggtccagctgggatacctggattccctggtatgaaaggacacagaggcttcgatggacgaaatggagaaaagggtgaaacaggtgctcctggattaaagggtgaaaatggtcttccaggcgaaaatggagctcctggacccatgggtccaagaggggctcctggtgagcgaggacggccaggacttcctggggctgcaggtgctcggggtaatgacggtgctcgaggcagtgatggtcaaccaggccctcctggtcctcctggaactgccggattccctggatcccctggtgctaagggtgaagttggacctgcagggtctcctggttcaaatggtgcccctggacaaagaggagaacctggacctcagggacacgctggtgctcaaggtcctcctggccctcctgggattaatggtagtcctggtggtaaaggcgaaatgggtcccgctggcattcctggagctcctggactgatgggagcccggggtcctccaggaccagccggtgctaatggtgctcctggactgcgaggtggtgcaggtgagcctggtaagaatggtgccaaaggagagcccggaccacgtggtgaacgcggtgaggctggtattccaggtgttccaggagctaaaggcgaagatggcaaggatggatcacctggagaacctggtgcaaatgggcttccaggagctgcaggagaaaggggtgcccctgggttccgaggacctgctggaccaaatggcatcccaggagaaaagggtcctgctggagagcgtggtgctccaggccctgcagggcccagaggagctgctggagaacctggcagagatggcgtccctggaggtccaggaatgaggggcatgcccggaagtccaggaggaccaggaagtgatgggaaaccagggcctcccggaagtcaaggagaaagtggtcgaccaggtcctcctgggccatctggtccccgaggtcagcctggtgtcatgggcttccccggtcctaaaggaaatgatggtgctcctggtaagaatggagaacgaggtggccctggaggacctggccctcagggtcctcctggaaagaatggtgaaactggacctcagggacccccagggcctactgggcctggtggtgacaaaggagacacaggaccccctggtccacaaggattacaaggcttgcctggtacaggtggtcctccaggagaaaatggaaaacctggggaaccaggtccaaagggtgatgccggtgcacctggagctccaggaggcaagggtgatgctggtgcccctggtgaacgtggacctcctggattggcaggggccccaggacttagaggtggagctggtccccctggtcccgaaggaggaaagggtgctgctggtcctcctgggccacctggtgctgctggtactcctggtctgcaaggaatgcctggagaaagaggaggtcttggaagtcctggtccaaagggtgacaagggtgaaccaggcggtccaggtgctgatggtgtcccagggaaagatggcccaaggggtcctactggtcctattggtcctcctggcccagctggccagcctggagataagggtgaaggtggtgcccccggacttccaggtatagctggacctcgtggtagccctggtgagagaggtgaaactggccctccaggacctgctggtttccctggtgctcctggacagaatggtgaacctggtggtaaaggagaaagaggggctccgggtgagaaaggtgaaggaggccctcctggagttgcaggaccccctggaggttctggacctgctggtcctcctggtccccaaggtgtcaaaggtgaacgtggcagtcctggtggacctggtgctgctggcttccctggtgctcgtggtcttcctggtcctcctggtagtaatggtaacccaggacccccaggtcccagcggttctccaggcaaggatgggcccccaggtcctgcgggtaacactggtgctcctggcagccctggagtgtctggaccaaaaggtgatgctggccaaccaggagagaagggatcgcctggtgcccagggcccaccaggagctccaggcccacttgggattgctgggatcactggagcacggggtcttgcaggaccaccaggcatgccaggtcctaggggaagccctggccctcagggtgtcaagggtgaaagtgggaaaccaggagctaacggtctcagtggagaacgtggtccccctggaccccagggtcttcctggtctggctggtacagctggtgaacctggaagagatggaaaccctggatcagatggtcttccaggccgagatggatctcctggtggcaagggtgatcgtggtgaaaatggctctcctggtgcccctggcgctcctggtcatccaggcccacctggtcctgtcggtccagctggaaagagtggtgacagaggagaaagtggccctgctggccctgctggtgctcccggtcctgctggttcccgaggtgctcctggtcctcaaggcccacgtggtgacaaaggtgaaacaggtgaacgtggagctgctggcatcaaaggacatcgaggattccctggtaatccaggtgccccaggttctccaggccctgctggtcagcagggtgcaatcggcagtccaggacctgcaggccccagaggacctgttggacccagtggacctcctggcaaagatggaaccagtggacatccaggtcccattggaccaccagggcctcgaggtaacagaggtgaaagaggatctgagggctccccaggccacccagggcaaccaggccctcctggacctcctggtgcccctggtccttgctgtggtggtgttggagccgctgccattgctgggattggaggtgaaaaagctggcggttttgccccgtattatggagatgaaccaatggatttcaaaatcaacaccgatgagattatgacttcactcaagtctgttaatggacaaatagaaagcctcattagtcctgatggttctcgtaaaaaccccgctagaaactgcagagacctgaaattctgccatcctgaactcaagagtggagaatactgggttgaccctaaccaaggatgcaaattggatgctatcaaggtattctgtaatatggaaactggggaaacatgcataagtgccaatcctttgaatgttccacggaaacactggtggacagattctagtgctgagaagaaacacgtttggtttggagagtccatggatggtggttttcagtttagctacggcaatcctgaacttcctgaagatgtccttgatgtgcatctggcattccttcgacttctctccagccgagcttcccagaacatcacatatcactgcaaaaatagcattgcatacatggatcaggccagtggaaatgtaaagaaggccctgaagctgatggggtcaaatgaaggtgaattcaaggctgaaggaaatagcaaattcacctacacagttctggaggatggttgcacgaaacacactggggaatggagcaaaacagtctttgaatatcgaacacgcaaggctgtgagactacctattgtagatattgcaccctatgacattggtggtcctgatcaagaatttggtgtggacgttggccctgtttgctttttataaaccaaactctatctgaaatcccaacaaaaaaaatttaactccatatgtgttcctcttgttctaatcttgtcaaccagtgcaagtgaccgacaaaattccagttatttatttccaaaatgtttggaaacagtataatttgacaaagaaaaatgatacttctctttttttgctgttccaccaaatacaattcaaatgctttttgttttatttttttaccaattccaatttcaaaatgtctcaatggtgctataataaataaacttcaacactctttatgataacaacactgtgttatattctttgaatcctagcccatctgcagagcaatgactgtgctcaccagtaaaagataacctttctttctgaaatagtcaaatacgaaattagaaaagccctccctattttaactacctcaactggtcagaaacacagattgtattctatgagtcccagaagatgaaaaaaattttatacgttgataaaacttataaatttcattgattaatctcctggaagattggtttaaaaagaaaagtgtaatgcaagaatttaaagaaatatttttaaagccacaattattttaatattggatatcaactgcttgtaaaggtgctcctcttttttcttgtcattgctggtcaagattactaatatttgggaaggctttaaagacgcatgttatggtgctaatgtactttcacttttaaactctagatcagaattgttgacttgcattcagaacataaatgcacaaaatctgtacatgtctcccatcagaaagattcattggcatgccacaggggattctcctccttcatcctgtaaaggtcaacaataaaaaccaaattatggggctgcttttgtcacactagcatagagaatgtgttgaaatttaactttgtaagcttgtatgtggttgttgatcttttttttccttacagacacccataataaaatatcatattaaaattc